(19) United States
(12) Patent Application Publication
(10) Pub. No.: US 2025/0352381 A1
(43) Pub. Date: Nov. 20, 2025

(54) Wearable Head-Neck Traction Braces with Two End-Effector Degrees-of-Freedom

(71) Applicant: The Trustees of Colombia University in the City of New York, New York, NY (US)

(72) Inventors: Sunil K. AGRAWAL, Newark, DE (US); Priya Kulkarni, New York, NY (US); Jingzong Zhou, New York, NY (US)

(73) Assignee: The Trustees of Colombia University in the City of New York, New York, NY (US)

(21) Appl. No.: 19/210,326

(22) Filed: May 16, 2025

Related U.S. Application Data
(60) Provisional application No. 63/648,979, filed on May 17, 2024.

(51) Int. Cl. A61F 5/048

(52) U.S. Cl. CPC A61F 5/048

(57) ABSTRACT

A number of alternative embodiments for implementing an articulated head-neck traction brace are described. Each of these embodiments has a base frame configured to be supported by a subject's shoulders, and an end-effector frame configured so that it can be removably attached to the subject's head, and either three or four chains of joints that run between the base frame and the end-effector frame. In some embodiments, each chain of joints has a Revolute-Prismatic-Universal-Revolute (RPUR) structure, and there are either three or four chains of joints. In other embodiments, each chain of joints has a Revolute-Revolute-Universal-Revolute (RRUR) structure, and there are four chains of joints.

### CROSS REFERENCE TO RELATED APPLICATIONS

This Application claims the benefit of U.S. Provisional Application 63/648,979, filed May 17, 2024, which is incorporated herein by reference in its entirety.

### BACKGROUND

Cervical spondylosis is a disease that refers to age-related degeneration of the cervical spine. This disk degeneration affects approximately 80-90% of people over the age of 50. Spondylosis can cascade into a variety of subsequent neck deformities, including cervical radiculopathy, which is a disorder caused by nerve root dysfunction. These deformities can cause weakness in the upper or lower extremities and difficulty with fine motor tasks. Furthermore, the pain and dysfunction associated with cervical disk degeneration can negatively affect quality of life and interfere with the ability to complete activities of daily living.

While surgery can correct some degenerative disk conditions, it increases the risk of complications. In order to mitigate the risks associated with surgery, nonoperative methods are used to treat cervical disk disease. A survey concluded that 93.1% of physical therapists use traction as a method for treating nerve root compression caused by cervical radiculopathy. In a clinical setting, manual and mechanical traction methods can be used to treat patients.

During manual traction, the patient's head and neck are manipulated by a trained physical therapist or a physician. The head is moved in a variety of orientations, and traction is applied as the clinician sees fit. Manual traction provides the clinician with the most freedom to manipulate the position and orientation of the head, but suffers from the error intrinsic to human manipulation. For example, when clinicians deliver traction forces manually within coarse categories of 0-20 N, 20-50 N, and 50 N+, they are able to achieve the correct level only 75% of the time.

Mechanical traction is another common method of applying cervical traction, where the patient lies in a supine position with the head flexed forward on a cradle. (As nomenclature, flexion and extension are rotational movements in the sagittal plane, where the chin is moving towards or away from the chest, respectively.) This cradle is attached to a machine that moves on a track in a direction away from the shoulders parallel to the cervical spine. Mechanical traction has an advantage over manual traction as it has more precise control over both the head position and the magnitude of forces applied during traction. It can also provide intermittent traction to the patient during which the traction force cycles between high and low values with specific timing. Intermittent traction has been shown to decrease pain in people with cervical radiculopathy and reduce the effect that the disease has on their activities of daily living. Intermittent traction has also been shown to outperform continuous traction in reducing pain and increasing mobility.

Yet another form of mechanical traction is over-the-door traction for at-home use. In this method, the user wears a head halter, which is fitted around the base of the head under the skull. The halter is connected to a water bag on a pulley, which provides an upward force on the head. In patient groups with cervical radiculopathy, over-door traction was found to lower neck disability and pain. But both forms of mechanical traction have limited control over the angle of the head, and neither allows for rotation in the frontal plane, also known as lateral bending.

Studies suggest that (a) mechanical intermittent cervical traction reduces pain; (b) application of upper cervical traction improved active cervical rotation and pain response; (c) mechanical traction force, with lateral bending, can improve cervical rotation range of motion and reduce neck pain; and (d) the range of motion for axial rotation, flexion/extension and lateral bending is significantly improved through cervical traction and exercise therapy.

Traction of the head-neck also plays an important role in the treatment of patients with neck pain. It has been noted that 26-71% of adults experience episodes of neck pain or stiffness in their lifetime. Self-reported neck problems contribute to large healthcare expenditures. The most common causes of neck pain are axial neck pain, whiplash-associated disorder (WAD), and cervical radiculopathy. A survey conducted by the American Physical Therapy Association (APTA) of 4,000 physical therapists who treat patients with neck pain concludes that physical therapists routinely use traction on patients for pain relief and comprehensive care.

Mechanical traction programs in neck pain treatment involve applying a traction force to the head-neck area by a machine in a specific direction. Intermittent Cervical Traction (ICT) is a common approach for mechanical traction. Patients who received ICT for neck pain had significantly lower pain scores than those who received placebos immediately after treatment. Another study with more than 100 participants shows that upper cervical traction also improves active cervical rotation. This study also suggests that mechanical traction, along with lateral bending of the head, can improve cervical rotation range of motion and reduce neck pain. The two forms of cervical treatment, mechanical traction and manual traction, are almost equally effective in reducing pain and increasing the range of motion.

### SUMMARY OF THE INVENTION

One aspect of this application is directed to a first head-neck traction brace. The first head-neck traction brace comprises a base frame, an end-effector frame, and four chains of joints. The base frame is configured to be supported by a subject's shoulders. The end-effector frame is configured so that it can be removably attached to the subject's head. Each of the four chains of joints has a lower revolute joint connected to the base frame followed by a prismatic joint followed by a universal joint followed by an upper revolute joint. The upper revolute joint is connected to the end-effector frame.

Some embodiments of the first head-neck traction brace further comprise a plurality of linear actuators, each of which is configured to actively control a respective one of the prismatic joints.

Some embodiments of the first head-neck traction brace further comprise two linear actuators, each of which is configured to actively control a respective lateralmost one of the prismatic joints. Optionally, in these embodiments, all of the prismatic joints that are not actively controlled by one of the linear actuators can be passive prismatic joints.

In some embodiments of the first head-neck traction brace, the universal joint comprises two revolute joints.

Some embodiments of the first head-neck traction brace further comprise at least one additional chain of joints having a first end connected to the base frame and a second end connected to the end-effector frame.

Another aspect of this application is directed to a second head-neck traction brace. The second head-neck traction brace comprises a base frame, an end-effector frame, and three chains of joints. The base frame is configured to be supported by a subject's shoulders. The end-effector frame is configured so that it can be removably attached to the subject's head. Each of the three chains of joints has a lower revolute joint connected to the base frame followed by a prismatic joint followed by universal joint followed by an upper revolute joint connected to the end-effector frame.

Some embodiments of the second head-neck traction brace further comprise a plurality of linear actuators, each of which is configured to actively control a respective one of the prismatic joints.

Some embodiments of the second head-neck traction brace further comprise two linear actuators, each of which is configured to actively control a respective lateralmost one of the prismatic joints. Optionally, in these embodiments, all of the prismatic joints that are not actively controlled by one of the linear actuators can be passive prismatic joints.

In some embodiments of the second head-neck traction brace, a plurality of revolute joints are used to implement each of the universal joints.

Some embodiments of the second head-neck traction brace further comprise a fourth chain of joints having a lower revolute joint connected to the base frame followed by a prismatic joint followed by universal joint followed by an upper revolute joint connected to the end-effector frame.

Another aspect of this application is directed to a third head-neck traction brace. The third head-neck traction brace comprises a base frame, an end-effector frame, and four chains of joints. The base frame is configured to be supported by a subject's shoulders. The end-effector frame is configured so that it can be removably attached to the subject's head. Each of the four chains of joints has a lower revolute joint that is mounted to the base frame followed by a middle revolute joint followed by a universal joint followed by an upper revolute joint that is mounted to the end-effector frame.

Some embodiments of the third head-neck traction brace further comprise a plurality of rotary motors, each of which is configured to actively control a respective one of the lower revolute joints.

Some embodiments of the third head-neck traction brace further comprise two rotary motors, each of which is configured to actively control a respective lateralmost one of the lower revolute joints. Optionally, in these embodiments, all of the lower revolute joints that are not actively controlled by one of the rotary motors can be passive revolute joints.

Some embodiments of the third head-neck traction brace further comprise at least one additional chain of joints having a first end connected to the base frame and a second end connected to the end-effector frame.

Another aspect of this application is directed to a fourth head-neck traction brace. The fourth head-neck traction brace comprises a base frame, an end-effector frame, and four chains of joints. The base frame is configured to be supported by a subject's shoulders. The end-effector frame is configured so that it can be removably attached to the subject's head. Each of the four chains of joints has a respective first end connected to the base frame and a respective second end connected to the end-effector frame. And each of the four chains of joints has either (a) a lower revolute joint followed by a prismatic joint followed by a universal joint followed by an upper revolute joint or (b) a lower revolute joint followed by a middle revolute joint followed by a universal joint followed by an upper revolute joint.

In some embodiments of the fourth head-neck traction brace, each of the lateralmost chains of joints has a lower revolute joint connected to the base frame followed by a prismatic joint followed by a universal joint followed by an upper revolute joint connected to the end-effector frame. These embodiments further comprise two linear actuators, each of which is configured to actively control a respective lateralmost one of the prismatic joints.

In some embodiments of the fourth head-neck traction brace, each of the lateralmost chains of joints has a lower revolute joint that is mounted to the base frame followed by a middle revolute joint followed by a universal joint followed by an upper revolute joint that is mounted to the end-effector frame. These embodiments further comprise two rotary motors, each of which is configured to actively control a respective lateralmost one of the lower revolute joints.

Some embodiments of the fourth head-neck traction brace further comprise at least one additional chain of joints having a first end connected to the base frame and a second end connected to the end-effector frame.

### BRIEF DESCRIPTION OF THE DRAWINGS

FIG. 1 depicts the structure and math model of a two degree-of freedom (2-DOF) neck brace in the neutral configuration.

FIG. 2 depicts the structure of each chain of the FIG. 1 embodiment.

FIG. 3 depicts the orientation of the head-neck in a fixed frame showing: (i) axial rotation, (ii) flexion and extension, and (iii) lateral bending.

FIG. 4 depicts two realizable solutions (I) and (II) of the inverse kinematics of the FIG. 1 embodiment.

FIG. 5 depicts two realizable solutions (I) and (II) of the forward kinematics of the FIG. 1 embodiment.

FIG. 6 depicts the workspace of the FIG. 1 embodiment when (I) rA=99.13 mm, (II) rA=20 mm. Two surfaces in each figure contain all positions that the end-effector can reach.

FIG. 7 depicts a fabricated prototype of the FIG. 1 embodiment.

FIG. 8 depicts (I) Z-translation and (II) lateral bending, validation results and desired values for the FIG. 1 embodiment.

FIG. 9 depicts component chain architectures with RPUR and RRUR joints.

FIG. 10 depicts the physical structure of a 4-RRUR spatial parallel neck brace embodiment.

FIG. 11 depicts the orientation of the head-neck in a fixed frame showing: (i) axial rotation, (ii) flexion and extension, and (iii) lateral bending.

FIG. 12 depicts a kinematics validation in SolidWorks and Python for the FIG. 10 embodiment.

FIG. 13 depicts avoiding solutions that may interfere with the human body and offer safety.

FIG. 14 depicts a workspace plot for a 4-chain-RPUR mechanism and a 4-chain-RRUR mechanism.

FIG. 15A depicts a SolidWorks model of the FIG. 10 embodiment.

FIG. 15B depicts a fabricated prototype of the FIG. 10 embodiment.

FIG. 16 depicts a control workflow for Vicon validation.

FIG. 17 depicts desired motion and captured motion for pure z-translation and z-translation with lateral bending for the FIG. 10 embodiment.

FIG. 18A is a Schematic diagram of another embodiment of a neck brace, with the base frame defined by the shoulders, with an origin at point O. The end-effector frame is at the head and has an origin at point P.

FIG. 18B is a schematic diagram of one chain of the FIG. 18A neck brace.

FIG. 19 depicts a CAD of the FIG. 18A brace with the chain 1 parts labeled.

FIG. 20 is a position control block diagram, where EE refers to the end-effector.

FIG. 21 depicts a force testing apparatus with a six-axis force-torque sensor installed between the brace end-effector and the fixed frame of the FIG. 18A neck brace.

FIG. 22 is a force control block diagram for the FIG. 18A neck brace.

FIG. 23 depicts command vs. expected position from forward kinematics for the FIG. 18A neck brace.

FIG. 24 depicts expected position from forward kinematics vs. actual position for the FIG. 18A neck brace.

FIG. 25 depicts a control block diagram, where EE refers to the end-effector.

FIG. 26 depicts a user wearing the neck brace during a traction experiment.

FIG. 27 depicts various positions of traction during experiments.

FIG. 28 depicts vertical force steady state values as measured by the load cells.

FIG. 29 depicts the angular error as computed by forward kinematics, in degrees. The top and bottom panels show the errors in the flexion-extension direction and the lateral bending direction, respectively.

FIG. 30 depicts the end-effector angular error, as measured by motion capture, in degrees. The top and bottom panels show the errors in the flexion-extension direction and the lateral bending direction, respectively.

FIG. 31 depicts the slip between the head and the end-effector as measured by motion capture, in degrees. The top and bottom panels show the slip in the flexion-extension direction and the lateral bending direction, respectively.

FIG. 32 depicts the activation of the Splenius Capitis muscle during steady-state force application.

Various embodiments are described in detail below with reference to the accompanying drawings, wherein like reference numerals represent like elements.

### DESCRIPTION OF THE PREFERRED EMBODIMENTS

This application is organized in four sections. Each of the first three sections describes a different type of head-neck brace; and the fourth section describes a validation study for the neck brace described in Section III.

Section I discloses embodiments in which an articulated head-neck traction brace includes a base frame and an end-effector frame, with four Revolute-Prismatic-Universal-Revolute (RPUR) structure chains disposed therebetween. Each of the universal joints can be implemented using a pair of revolute joints, in which case each of the four chains will have a Revolute-Prismatic-Revolute-Revolute-Revolute (RPRRR) structure.

Section II discloses embodiments in which an articulated head-neck traction brace includes a base frame and an end-effector frame, with four Revolute-Revolute-Universal-Revolute (RRUR) structure chains disposed therebetween.

Section III discloses embodiments in which an articulated head-neck traction brace includes a base frame and an end-effector frame, with three Revolute-Prismatic-Universal-Revolute (RPUR) structure chains disposed therebetween.

Section IV describes a validation study for the head-neck brace described in Section III.

In alternative embodiments (not shown), a mixture of RPUR chains and RRUR chains can be used. For example, the two lateralmost chains of joints could be RPUR chains in which the prismatic joint is driven by a respective linear actuator, and the remaining chains of joints could be RRUR chains. Or in another example, the two lateralmost chains of joints could be RRUR chains in which the lowest revolute joint is driven by a respective rotary motor, and the remaining chains of joints could be RPUR chains.

### Section I

### 1. Introduction

This section I describes a parallel-actuated robotic mechanism designed to provide two degrees-of-freedom (DOF) to the end-effector relative to a fixed base. In a potential application as a head-neck traction brace, these two independent DOFs are the vertical translation of the head with respect to shoulders and a specified orientation of the head in lateral bending. Motivated by recommended clinical methods to apply traction forces on the head, it is designed to provide vertical traction force on the head while tilted in a specific orientation.

The design has four component chains starting from a base stationed at the shoulders, each chain having 5 DOFs. Each chain imposes a single constraint on the motion of the end-effector. Together, four chains would apply four constraints, allowing only two DOFs of motion to the end-effector. Two out of four component chains are actively driven by linear actuators. Our kinematic studies show that the achievable workspace of this mechanism with a specific stroke length of actuators of ±50 mm results in 175-222 mm of vertical translation and up to ±9° of lateral bending. The lateral bending is coupled to the flexion/extension angle of the end-effector. A physical prototype was constructed to investigate the functional realization of the design in hardware. Overall, the physical prototype validated the motion of the theoretical model despite potential errors in the fabrication, making the design a good candidate for providing head-neck traction.

Existing treatment methods for cervical traction have several limitations: (i) lack of control of the head posture relative to the shoulders when traction force is applied, (ii) poor control of the traction force over time, and (iii) inability to perform other functions when participants lie on a bench and undergo traction. The limitations of existing cervical traction methods may be remedied through the use of robotic neck braces. But existing robotic neck braces lack the ability to provide an independent z-translation motion while also being able to position the head in lateral bending and flexion.

In contrast, the embodiments described in this section provide 2 DOF at the end-effector and uses a 4-chain parallel mechanism. The architecture of these embodiments has been specifically chosen to provide vertical translation to the head relative to the shoulders while the neck is in a specific orientation. The mechanism's two controllable DOFs are the vertical translation and lateral bending angle. With this choice of the mechanism, the vertical translation DOF can be used to control the traction force and the lateral bending would be used to select the head orientation of the patient with cervical spondylosis.

### 2. Mathematical Model

### A. Physical Structure

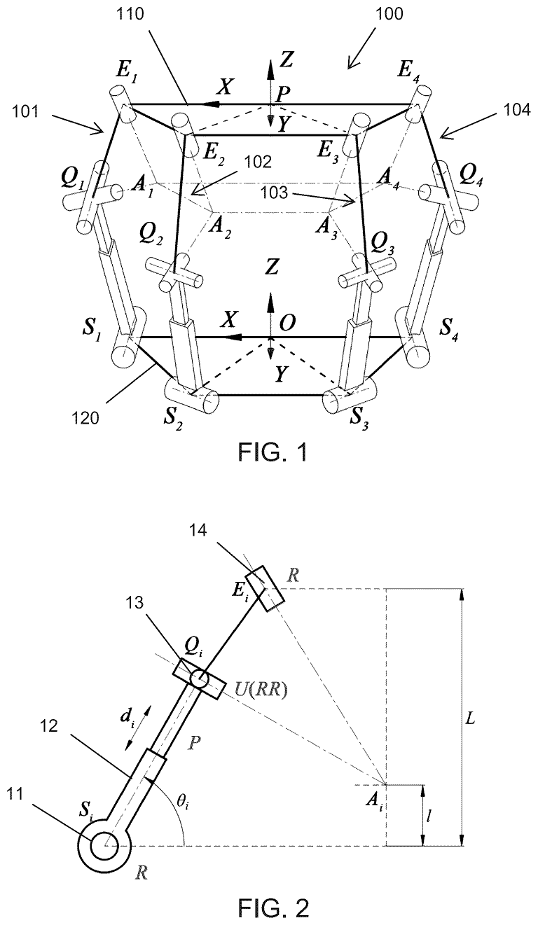

FIGS. 1, 2, and 4 depict one embodiment of a robotic neck brace 100 that has a base frame 120 (F_{O}) that is configured to be supported by the subject's shoulder, an end-effector 110 (F_{P}) configured so that it can be removably attached to the subject's head (e.g., using a headband positioned at the subject's forehead), and four chains of joints 101-104. The points S₁, Q₁, A₁ within chain 101 and points S₄, Q₄, A₄ within chain 104 are constrained to lie within the XOZ plane of the base frame 120. Similarly, points S₂, Q₂, A₂ within chain 102 lie on a vertical plane at ∠S₁OS₂=60° to the X-axis. Similarly, the points S₃, Q₃, A₃ within chain 103 lie on a vertical plane at ∠S₁OS₃=120° to the X-axis.

Each of the four chains 101-104 has a RPUR structure. Thus, beginning at the base frame 120 and moving up towards the end-effector 110, the order of the joints is as follows: a lower revolute joint 11 that is connected to the base frame 120, followed by a prismatic joint 12, followed by a universal joint 13, followed by an upper revolute joint 14. The upper revolute joint 14 is connected to the end-effector frame 110. Sᵢ and Eᵢ are revolute (R) joints, Sᵢ and Qᵢ are connected by a prismatic joint (P), Qᵢ is a universal (U) joint consisting of two revolute joints. L, l are the heights of the end-effector and the plane containing A relative to the fixed base frame 120, respectively.

The axis of the revolute joint located at Sᵢ and the axis of the lower revolute joint located at Qᵢ within the universal joint in the chain are parallel. The axis of the second revolute joint within the universal joint located at Qᵢ and the axis of the revolute joint located at Eᵢ intersect at a point Aᵢ. The points Sᵢ, Qᵢ, and Aᵢ are coplanar within each chain. The axis of the prismatic joint 12 is normal to the first pair of revolute joints. The points S₁, S₂, S₃, S₄ are located on a semi-circle with a radius of rₛ and sit on the fixed shoulder of the human user, considered as the fixed global frame. The intersection points A₁, A₂, A₃, A₄ are shown in FIG. 1.

In the neutral configuration, these points are chosen to lie on a circle of radius r_{A} that form a virtual intermediate plane that is parallel to the end-effector plane and the base plane. With this choice in the neutral configuration, the plane formed by the intersection points A₁, A₂, A₃, A₄ will remain parallel to the plane formed by the intersection points E₁, E₂, E₃, E₄. In the physical design of the mechanism, the stroke lengths of both linear actuators are 25 mm.

Referring now to FIG. 4, the prismatic joints 12 on chains 101 and 104 are actively controlled by respective linear actuators 18, while the prismatic joints 12 on chains 102 and 103 are passive. d₁, d₄ are stroke lengths of linear actuated joints, while d₂, d₃ are stroke lengths of passive prismatic joints. Thus, as best seen in FIG. 4(I), there are two linear actuators 18, each of which is configured to actively control a respective lateralmost one of the prismatic joints 12. All of the prismatic joints 12 that are not actively controlled by one of the linear actuators 18 are passive prismatic joints. Optionally, at least one additional chain of joints (not shown) having a first end connected to the base frame 120 and a second end connected to the end-effector frame 110 can be added. The universal joint 13 can be implemented using two revolute joints.

### B. Description of the End-Effector Motion

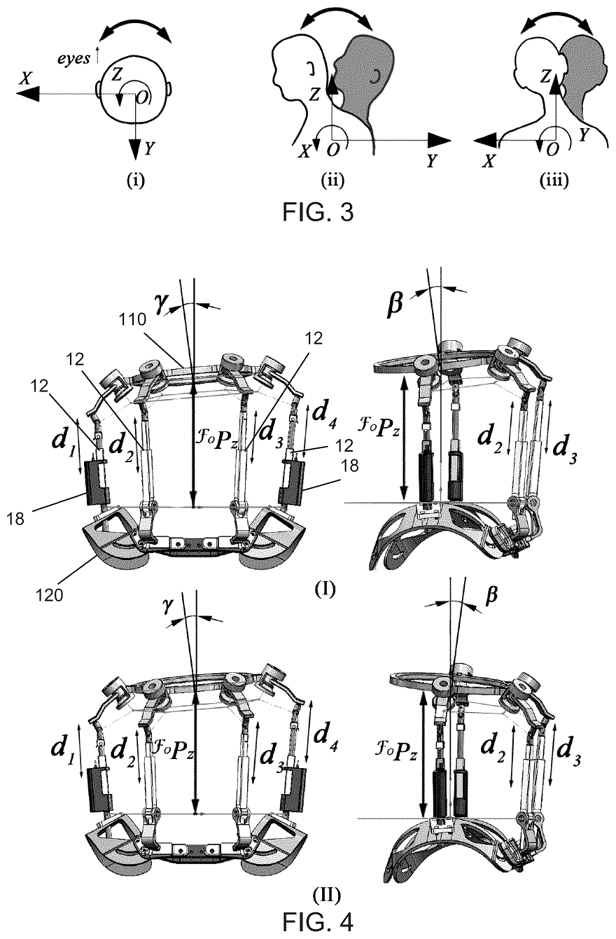

The three orientation angles are described in the fixed coordinate frame in FIG. 3. A space-fixed 3-1-2 angle sequence is selected to describe the orientation of the end-effector frame F_{P} relative to the base frame F_{O}. This rotation matrix is given in Eq. (1), where sin β is simplified as s_{β}, cos β is simplified as c_{β} and so on. Axial rotation, flexion/extension, and lateral bending are denoted as α, β, and γ.

$${}_{}^{O} {R}_{P}^{} = [ \begin{matrix}{s}_{α} {s}_{β} {s}_{γ} + {c}_{γ} {c}_{α} & {c}_{α} {s}_{β} {s}_{γ} - {c}_{γ} {s}_{α} & {c}_{β} {s}_{γ} \\ {s}_{α} {c}_{β} & {c}_{α} {c}_{β} & - {s}_{β} \\ {s}_{α} {s}_{β} {c}_{γ} - {s}_{γ} {c}_{α} & {c}_{α} {s}_{β} {c}_{γ} + {s}_{γ} {s}_{α} & {c}_{β} {c}_{γ}\end{matrix} ] \qquad ( 1 )$$

The next step is to express the coordinates of E₁, . . . , E₄ and A₁, . . . , A₄ which are fixed points in the end-effector frame F_{P} in the fixed base frame F_{O}, as shown in Eqs. (2) and (3). The symbols for the points in the end-effector frame F_{P} are ^{F}^{_{P}}Eᵢ or ^{F}^{_{P}}Aᵢ, i=1, . . . , 4. The coordinates of these points in the base frame F_{O} are given below, where ^{F}^{_{O}}P is the coordinate of the origin which lies at the midpoint of E₁E₄ on the end-effector frame F_{P}.

$${}^{{ℱ}_{O}} {E}_{i} = {}^{{ℱ}_{O}} P + {}_{}^{O} {R}_{P}^{} {}^{{ℱ}_{P}} {E}_{i} , i = 1 , … , 4 \qquad ( 2 )$$

$${}^{{ℱ}_{O}} {A}_{i} = {}^{{ℱ}_{O}} P + {}_{}^{O} {R}_{P}^{} {}^{{ℱ}_{P}} {A}_{i} , i = 1 , … , 4 \qquad ( 3 )$$

From the specific constraint on the geometry, A₁ and A₄ are limited to XOZ plane, i.e. the y-coordinates of these points are zero and satisfy Eqs. (4) and (5).

$${}^{{ℱ}_{O}} {A}_{1 y} = {}^{{ℱ}_{O}} {P}_{y} + {s}_{α} {c}_{β} {r}_{A} + {s}_{β} h = 0 \qquad ( 4 )$$

$${}^{{ℱ}_{O}} {A}_{4 y} = {}^{{ℱ}_{O}} {P}_{y} - {s}_{α} {c}_{β} {r}_{A} + {s}_{β} h = 0 \qquad ( 5 )$$

From Eqs. (4) and (5), since r_{A}≠0, if c_{β}≠0, the term sₐ should be zero, i.e., a=0. In other words, the term sₐc_{β}r_{A} in Eq. (4) and sₐc_{β}r_{A} in Eq. (5) should be zero. This implies that the axial rotation of the end-effector is zero. If c_{β}=0, it implies that the lateral ₂ bending of the end-effector is π/2. In general, this specific pose of the end-effector, i.e., the head relative to the shoulder, with β=π/2, is unreachable due to geometric constraints. Thus, the rotation matrix (1) can be simplified with the condition a=0.

Additionally, ^{F}^{_{O}}P_{y}+s_{β}(Ll)=0. The geometric constraints on the locations of A₂ and A₃ create Eqs. (6) and (7):

$${}^{{ℱ}_{O}} {A}_{2 y} = tan 60 ° {}^{{ℱ}_{O}} {A}_{2 x} \qquad ( 6 )$$

$${}^{{ℱ}_{O}} {A}_{2 y} = tan 120 ° {}^{{ℱ}_{O}} {A}_{3 x} \qquad ( 7 )$$

From the above equations, on simplification, we get

$${c}_{β} = {c}_{γ} \qquad ( 8 )$$

According to Eq (8), the flexion/extension motion and lateral bending are coupled together as β=γ or β=γ. Based on such relationship, other end-effector positions ^{F}^{_{O}}Pₓ, ^{F}^{_{O}}P_{y} can then be determined with γ and ^{F}^{_{O}}P_{z} chosen independently.

### C. Inverse Kinematics

The inverse kinematics problem is to find the displacement of each prismatic joint given the position and orientation of the end-effector. As described in section I-2-B, for an end-effector with 2 DOF, one can choose the following two independent variables γ, ^{F}^{_{O}}P_{z} to describe the position and orientation of the end-effector and the remaining end-effector position/orientation variables will be determined.

The lengths of prismatic joints can be solved by the distance equations given in Eqs. (9) and (10):

$${‖ {}^{{ℱ}_{O}} {A}_{i} - {}^{{ℱ}_{O}} {Q}_{i} ‖}^{2} = {‖ {A}_{i} {Q}_{i} ‖}^{2} , i = 1 , … , 4 \qquad ( 9 )$$

$${‖ {}^{{ℱ}_{O}} {E}_{i} - {}^{{ℱ}_{O}} {Q}_{i} ‖}^{2} = {‖ {E}_{i} {Q}_{i} ‖}^{2} , i = 1 , … , 4 \qquad ( 10 )$$

These distance equations will result in Eqs. (11) and (12) with the following structure:

$${f}_{{A}_{i} {Q}_{i}} ( {d}_{i} , {θ}_{i} ) = 0 , i = 1 , … , 4 \qquad ( 11 )$$

$${f}_{{E}_{i} {Q}_{i}} ( {d}_{i} , {θ}_{i} ) = 0 , i = 1 , … , 4 \qquad ( 12 )$$

In total, there will be 8 equations involving 8 variables as described in Eqs. (11) and (12). Solutions to these equations can be resolved e.g., in the scipy numerical solver of Python 3.9.

Sample Solutions: The solution sets for d₁, . . . , d₄ representing S₁Q₁E₁, . . . , S₄Q₄E₄ using Eqs. (11) and (12) for each chain were computed for an example end-effector pose: γ=5°, ^{F}^{_{O}}P_{z}=200 mm. For each configuration γ=β and γ=β, we obtained 16 real solutions of the joint variables. Each chain yielded two real solutions of the stroke length dᵢ, which together resulted in 2×2×2×2=16 solutions.

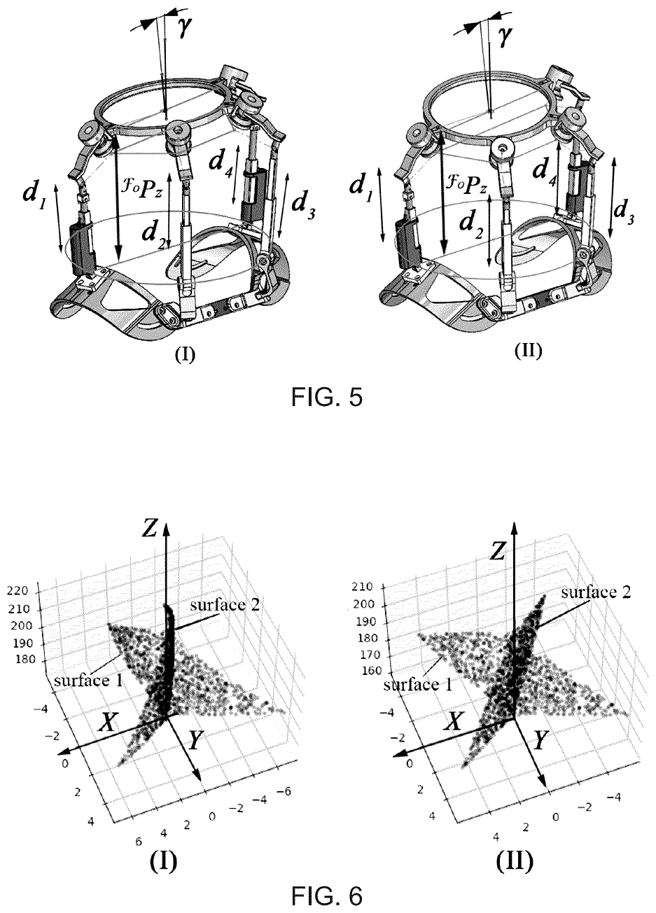

However, these 16 solutions are not all physically realizable as each chain can be in an “arm up” or an “ARM down” configuration. Also, there are physical limits on the range of motion, i.e., the stroke length of each dᵢ. Once any chain has been assembled in an ‘arm up’ or an ‘arm down’ configuration, the stroke lengths on the chains may not be allowed to flip between the two configurations. Hence, out of the 16 potential solutions from the inverse kinematics that we obtained for γ=β and γ=β, we present the two solutions that are physically realizable within the structure of the neck brace assembly. Each of these two configurations is shown in a 3D geometrical model in FIG. 5. The corresponding numerical solutions are listed in Table I.

TABLE I
JOINT VARIABLES AND POSE VARIABLES
IN FIG. 4 (UNIT: MM)

| Sol. No. | γ | [symbol] P [symbol] | β | d₁ | d [symbol] | d₂ | d [symbol] |
|---|---|---|---|---|---|---|---|
| (I) | 5° | 200 | 5° | 13.70 | 41.02 | 33.67 | 47.54 |
| (II) | 5° | 200 | −5° | 13.70 | 41.02 | 10.45 | 23.75 |

[symbol]

indicates data missing or illegible when filed

The first column of Table I represents the names of the solutions in FIG. 5. The second and third columns in Table I list the end-effector pose. The last four columns are the solutions of the joint variables when β=γ and β=−γ.

If the input parameter γ=0°, there will be only one solution for inverse kinematics. Otherwise, for a specific input when γ=0°, the computations are such so that there are two feasible solutions.

### D. Forward Kinematics

Forward kinematics computes the end-effector position and orientation given the joint displacements. Here, the inputs provided are stroke lengths from chain S Q E and chain S₄Q₄E₄, two equations related to bending angle γ are:

$${f}_{1} ( {}^{{ℱ}_{O}} {P}_{x} , {}^{{ℱ}_{O}} {P}_{y} , {}^{{ℱ}_{O}} {P}_{z} , γ , {d}_{1} ) = 0 \qquad ( 13 )$$

$${f}_{2} ( {}^{{ℱ}_{O}} {P}_{x} , {}^{{ℱ}_{O}} {P}_{y} , {}^{{ℱ}_{O}} {P}_{z} , γ , {d}_{4} ) = 0 \qquad ( 14 )$$

Eqs. (13) and (14) are obtained from chain S₁Q₁E₁ and S₄Q₄E₄ and they contain the variables ^{F}^{_{O}}Pₓ, ^{F}^{_{O}} P_{y}, ^{F}^{_{O}}P_{z}, β and γ.

In addition, we know that there are two branches of solution β=γ or β=γ. On substituting these (shown in Eqs. (4), (5), (6) and (7)), there remain only two independent unknowns ^{F}^{_{O}}P_{z}, γ. The coupled motion of flexion/extension and lateral bending are not limitations since the traction will happen almost quasi-statically.

In order to verify the computations, we chose the same input stroke-lengths as in Table 1, i.e., d₁=13.70 mm, d₄=41.02 mm. We found that there are four solutions when γ=β and another four when γ=β. Considering again the practical assembly of the solutions, we narrowed these down to two solutions which are listed in Table II and plotted in FIG. 5. These are technically the same solutions in inverse kinematics shown in FIG. 4.

The forward kinematic solutions yield the same solutions as in the inverse kinematics. This shows that the results of inverse and forward kinematics validate each other.

TABLE II
JOINT VARIABLES AND POSE VARIABLES
IN FIG. 5 (UNIT: MM)

| Sol. No. | d₂ | d [symbol] | γ | β | [symbol] P [symbol] | [symbol] P [symbol] | [symbol] P [symbol] |
|---|---|---|---|---|---|---|---|
| (I) | 33.67 | 47.54 | 5° | 5° | 1.87 | −2.53 | 200 |
| (II) | 10.45 | 23.75 | 5° | −5° | 3.17 | 2.53 | 200 |

[symbol]

indicates data missing or illegible when filed

### E. Workspace

The workspace of the FIG. 1 embodiment is shown for two sets of parameters in FIG. 6: (i) r_{A}=99.13 mm, and (ii) r_{A}=20 mm. We chose r_{A} as a parameter to vary in the computation of feasible workspace. Other parameters that were used to compute the workspace are shown in Table III.

TABLE III
PARAMETERS IN TWO DIFFERENT DESIGNS (UNIT: MM)

| Design | r_{A} | L | L − l | d₁ | d₄ |
|---|---|---|---|---|---|
| (i) | 99.13 | 197.68 | 29.01 | 25 | 25 |
| (ii) | 20 | 197.68 | 29.01 | 25 | 25 |

In Table III, the parameter L is measured when d₁ and d₄ are at half their stroke lengths which is 25 mm. This parameter is kept the same in (i) and (ii). Additionally, L l is kept the same for both designs (i) and (ii). For these two different parameters, the dimension of QᵢEᵢ are different and results in different workspaces.

FIG. 6 shows the points where the origin of the reference frame on the end-effector can reach (i.e., it shows the end-effector workspace). As r_{A} becomes larger, the two surfaces become more curved. A systematic study can be performed to characterize the workspace as needed in the future.

### 3. Validation with a Prototype

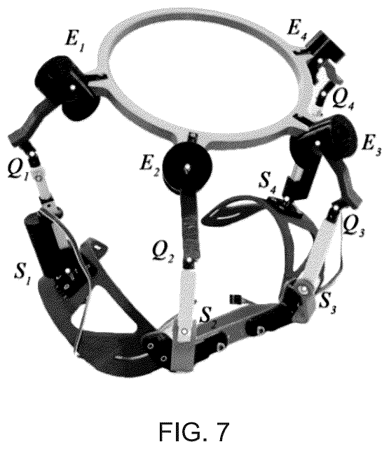

One example of a fabricated prototype is shown in FIG. 7 with main dimensions shown in Table IV.

TABLE IV
DIMENSIONS OF THE PROTOTYPE (UNIT: MM)

| E₁E₄ | S₁S₄ | Q₁S₁, Q₄S₄ | Q₂S₂, Q₃S₃ | QᵢEᵢ |
|---|---|---|---|---|
| 240 | 282 | 117.635 | 116.635 | 65.36 |

Except for linear actuators, universal joints, screws and bolts, all other parts were fabricated using a 3D printer. After the assembly of parts, two ACTUONIX P16 micro linear actuators were connected to a particle photon circuit board (which included a WiFi module) and were actuated. Of course, other appropriate actuators may be substituted for the ACTUONIX P16. A Vicon camera system was used to validate the motion of the end-effector after implementing the commands to the linear actuators.

The process of Vicon validation is described as 4 steps: (i) python GUI sends the commands, (ii) photon parses the commands and sends those to linear actuators, (iii) end-effector of the neck brace is driven by linear actuators, (iv) vicon cameras capture the motion of the end-effector. The tests are done with sinusoidal motion control. The sinusoidal motion is a continuous motion command to the end-effector for lateral bending and z translation. After getting the commands, the Photon controls the linear actuators to move the end-effector of the neck brace through continuous motion.

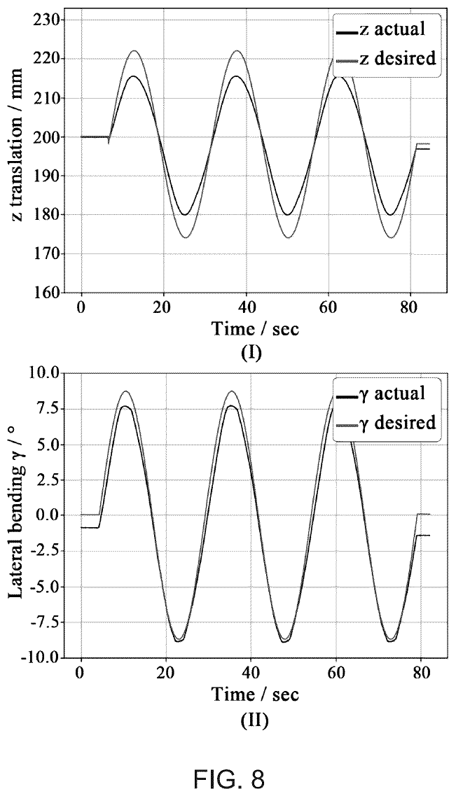

In order to know how well the system performs when following the commands, Vicon markers are placed on both the end-effector and shoulder base pad to validate the end-effector orientation and position. The motion of the end-effector is captured by Vicon Cameras and processed using Vicon Nexus software. The results from the sinusoidal motion are shown in FIG. 8.

Table IV shows the major dimensions in the fabricated prototype. The distance S₁S₄ is selected to be twice the distance of rₛ. The distance E₁E₄ is twice the distance of r_{E}. Distances for QᵢSᵢ are measured when they are not actuated. The dimension of E₁E₄ is based on the average human head size and the dimension S₁S₄ on the average size of human shoulders. Angles between QᵢSᵢ and plane S₁S₂S₃S₄ (base plane) are always larger than 90° as this is more ergonomic when participants wear the neck brace.

FIG. 8 shows the plots of the measured z-translation and lateral bending angle, distance and the commanded values. Black lines are the measured data, while the red lines are the commanded data to the mathematical model. The errors between the desired commands and the measurement from Vicon are presented in Table V. These results show a close match between the motion of the physical prototype and the mathematical model.

TABLE V
POSITION CONTROL ERRORS

| | lateral | z translation |
|---|---|---|
| | bending γ/° | ^{^{F}}^{O}P_{z}/mm |
| Mean Errors | 0.17 | 3.86 |
| RMS Errors | 0.84 | 4.42 |

The differences in the features of the physical prototype and the assumptions in the mathematical model likely caused the errors observed in the position validation. Small fabrication errors, such as rotation inherent to the linear actuators, flexibility of the 3D printed links, and slight backlash in the joints can contribute to cumulative errors in the end-effector motion. Despite these unavoidable manufacturing errors, the overall motions in lateral bending and z-translation of the end-effector follow the desired motion, as shown in FIG. 8. The current 4-chain parallel embodiments with 2-DOF of end-effector motion can provide vertical translation and lateral bending to the head-neck area. The control method in the current design is only limited to position control whereas force control is not implemented. These embodiments allow for a range of 9° to 9° lateral bending, which is adequate to automate traction provided by physical therapists. As discussed in the introduction, mechanical traction force with lateral bending can reduce neck pain and the device 100 can provide such a motion.

### 4. Conclusion

This section I describes the design of four-chain parallel embodiments that provide 2-DOF motion to the end-effector. These embodiments advantageously provide traction force on the head-neck along vertical direction while orienting the head to provide lateral bending. We characterized its inverse and forward kinematics. We fabricated a prototype to show the physical realizability of this design and how potential manufacturing errors can impact its performance. The prototype followed the mathematical model quite well despite potential errors in fabrication. This design opens up new possibilities for use in medical applications relating to the head-neck.

### Section II

### 1. Nomenclature

Table I lists nomenclature used in this section II.

TABLE 1

| | |
|---|---|
| L | Distance from base frame to the end-effector |
| | frame at neutral configuration |
| l | Distance from base frame to the intermediate |
| | frame at neutral configuration |
| R | Revolute joint |
| P | Prismatic joint |
| U | Universal joint |
| F | Frame symbol |
| α | Z-axis rotation angles/axial rotation angles |
| β | Flexion/extension angles |
| γ | Lateral bending angles |
| θ | Angles of the link SᵢKᵢ relative to base plane |
| ϕ | Angles of the K joint on each chain |
| i | Chain number or joint number, depending |
| | on the context |
| x | x component of the coordinate |
| y | y component of the coordinate |
| z | z component of the coordinate |
| P | End-effector frame |
| A | Intermediate plane |
| ^{O}R_{P} | Rotation matrix from base frame to |
| | end-effector frame |
| ^{^{F}}^{O}p | Coordinate of the end-effector origin |
| | in base frame F_{O} |
| s_{θ} | sin θ |
| c_{θ} | cos θ |

### 2. Introduction

Traction of the head-neck is important in the treatment of patients suffering from neck pain due to degeneration of the intervertebral discs. Conventional neck traction is provided manually by experienced physical therapists who maintain a desired orientation of the head-neck relative to the trunk while applying the traction. It is postulated that innovative designs of neck exoskeletons can provide the same function both flexibly and accurately. This section presents a novel architecture of a parallel mechanism whose base sits on the human shoulders with 4 parallel chains, each chain having a Revolute-Revolute-Universal-Revolute (RRUR) structure, while the end-effector is connected rigidly to the human head.

Each chain has five degrees-of-freedom (DOF) and applies a constraint on the motion of the end-effector. As a result, this parallel mechanism allows two DOFs to the end-effector. These are (i) forward flexion or lateral bending of the head, and (ii) vertical translation. An important motivation for the current design with the RRUR structure is to characterize the range of forward flexion/lateral bending of the head-neck with this structure and the vertical translation to the end-effector. A physical prototype was constructed and tested to evaluate the performance of this mechanism in hardware for the proposed application.

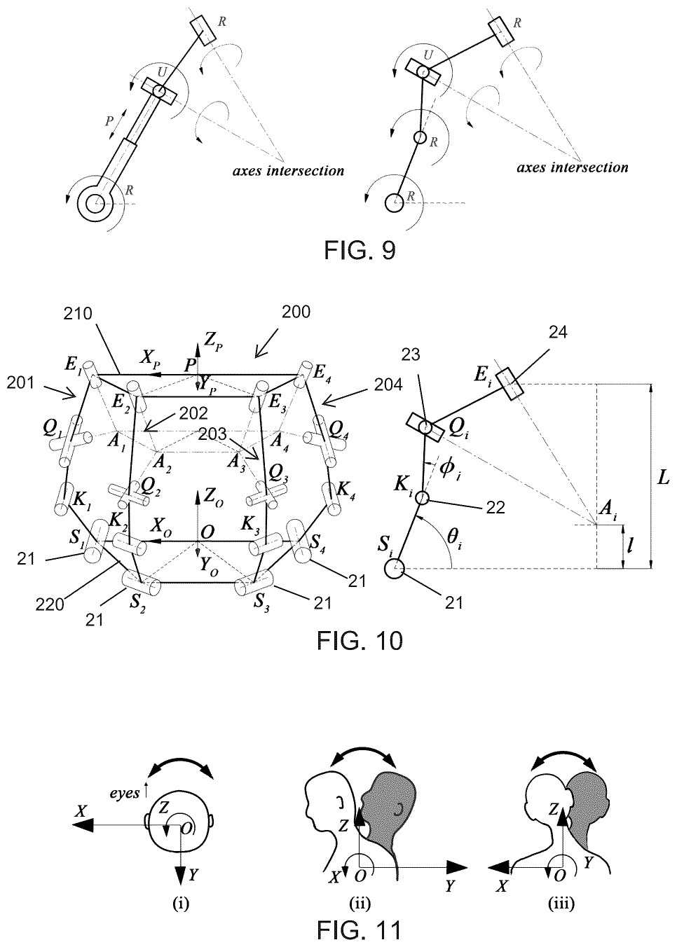

In clinical research, traction plays an important role in the treatment of patients with neck pain. Thus, the design of the neck brace focuses on applying traction to the head-neck along with a single degree-of-freedom rotation. In this section II, we analyze a 4-chain-RRUR 2-DOF parallel mechanism for a neck brace to provide traction, where the structures of the component chains are as shown on the right side of FIG. 9. We also compare the workspace of this mechanism with a 4-chain-RPUR mechanism, whose component chains are shown on the left side of FIG. 9. Both mechanisms can provide independent control of the vertical translation and lateral bending of the end-effector with respect to the base. Considering that during manual traction, therapists often prefer to tilt the head away from the vertical configuration, this section describes a parallel mechanism to provide traction force while allowing a rotation to the head relative to the shoulders. This mechanism is a wearable neck brace with 2 DOFs, which can be used to selectively apply controlled vertical traction on the head while achieving specific head orientation.

### 3. The Mathematical Model and Kinematic Analysis

### 3.1 Physical Structure

FIG. 10 depicts an embodiment of a neck brace 200 that includes four chains of joints 201-204, a rigid end-effector 210 with attachment point E₁, . . . , E₄, and a base frame 220 with attachment points for revolute joints S₁, . . . , S₄. The base frame 220 is configured to be supported by a subject's shoulders, and the end-effector 210 is configured so that it can be removably attached to the subject's head. Each of the chains 201-204 has a lower revolute joint 21 that is mounted to the base frame 220, followed by a middle revolute joint 22, followed by a universal joint 23, followed by an upper revolute joint 24 that is mounted to the end-effector frame 210. The universal joint can be constructed using two revolute joints, in which case each chain will be composed of 5 revolute joints. Two out of the four lower revolute joints 21, i.e., the lateralmost ones S₁, S₄ are activated by rotary motors, with each of the rotary motors configured to actively control a respective lateralmost one of the lower revolute joints 21. The two remaining lower revolute joints 21 S₂, S₃ are passive revolute joints. Joints K₁, . . . , K₄ and E₁, . . . , E₄ are passive revolute joints (and are not actively controlled by any of the rotary motors).

Joints Q₁, . . . , Q₄ are universal joints, implemented e.g., as a combination of two revolute joints. For each chain SᵢKᵢQᵢEᵢ in the mechanism, the axes of the revolute joints Sᵢ, Kᵢ, and Qᵢ are parallel. The axis of the second revolute joint making up the universal joint at Qᵢ intersects the axis of the revolute joint Eᵢ at the point Aᵢ. These intersection points are chosen to make up an intermediate plane A₁A₂A₃A₄. The two chains S₁K₁Q₁ and S₄K₄Q₄ are arranged within the plane X₀OZ₀ such that the revolute joints S₁, S₄ are oriented along the yₒ axis. The chain S₂K₂Q₂ is placed on the base plane with an angle ∠S₁OS₂=60°. Similarly, chain S₃K₃Q₃ is placed on the base plane with an angle ∠S₁OS₃=120°. Furthermore, joint variables on revolute joints S₁, . . . , S₄ are labeled θ₁, . . . , θ₄, and joint variables on revolute joints K₁, . . . , K₄ are denoted by ϕ₁, . . . , ϕ₄. One can choose S₁S₂S₃S₄ to lie on tangents to a circle with the origin at O, A₁A₂A₃A₄ will also form a circle with the origin at the midpoint of A₁A₄, and E₁E₂E₃E₄ a circle with the origin being P. L and l are respectively the heights of the end-effector and the virtual plane containing Aᵢ relative to the fixed base frame X₀Y₀Z₀.

Each chain shown in FIG. 10 has 3 revolute joints plus a universal joint (which can be made using two more revolute joints). Together, these add up to 5 degrees-of-freedom, thus providing one constraint to the motion of the end-effector. In total, 4 chains SᵢKᵢQᵢEᵢ provide 4 constraints on the motion of the end-effector. With four constraints on the motion of the end-effector, the end-effector has overall two degrees-of-freedom of motion.

### 3.2 Description of the End-Effector Motion

The human head has both translation and rotation degrees-of-freedom with respect to the shoulders. However, the translational range of motion of the head is small and the translation is often coupled with rotation. In our design, the end-effector will be attached to the human head. While we will do a more formal analysis of the motion of the end-effector in the upcoming portions of this section II, intuitively, due to the placements of the chains S₁K₁Q₁ and S₄K₄Q₄ with specific orientation of the lower revolute joints within these chains, the points A₁ and A₄ are restricted to the plane X₀OZ₀. As a result, the end-effector will have no axial rotation. We will expand on this derivation mathematically in the next portion of this section.

### 3.3 Inverse Kinematics Analysis

The inverse kinematics problem for this 4-chain-RRUR mechanism is to find the joint angles for each chain given the position and orientation of the end-effector. The following steps describe how to find the solution for inverse kinematics. Let [symbol] ₀ represent the base frame X₀Y₀Z₀, [symbol] represent the x, y, and z coordinates of P in the frame [symbol] ₀. Similarly, [symbol] _{P} represents end-effector frame X_{P}Y_{P}Z_{P}. A symbol such as [symbol] represents the coordinates of E₁ in frame [symbol] _{P}.

Step 1: We need to choose a rotation sequence that will describe the orientation of the end-effector frame in the base frame. Here, we choose the Space 3-1-2 rotation sequence with the three successive angles α, β, γ around Z, X, and Y axes. Here, α is the axial rotation or Z-axis rotation, β is the flexion/extension angle, and γ is the lateral bending angle.

$${}_{}^{O} {R}_{P}^{} = [ \begin{matrix}{s}_{α} {s}_{β} {s}_{γ} + {c}_{γ} {c}_{α} & {c}_{α} {s}_{β} {s}_{γ} - {c}_{γ} {s}_{α} & {c}_{β} {s}_{γ} \\ {s}_{α} {c}_{β} & {c}_{α} {c}_{β} & - {s}_{β} \\ {s}_{α} {s}_{β} {c}_{γ} - {s}_{γ} {c}_{α} & {c}_{α} {s}_{β} {c}_{γ} + {s}_{γ} {s}_{α} & {c}_{β} {c}_{γ}\end{matrix} ] \qquad ( 1 )$$

Step 2: Describe points E₁, . . . , E₄ and A₁, . . . , A₄ in end-effector frame [symbol] _{P} as [symbol] ₁, . . . , [symbol] ₄ and [symbol] ₁, . . . , [symbol] ₄.

Step 3: Convert [symbol] ₁, . . . , [symbol] ₄ and [symbol] ₁, . . . , [symbol] ₄ to base frame [symbol] ₀ using the following equations:

$${}^{{ℱ}_{O}} {A}_{i} = {}^{{ℱ}_{O}} P + {}_{}^{O} {R}_{P}^{} {}^{{ℱ}_{P}} {A}_{i} \qquad ( 2 )$$

$${}^{{ℱ}_{O}} {E}_{i} = {}^{{ℱ}_{O}} P + {}_{}^{O} {R}_{P}^{} {}^{{ℱ}_{P}} {E}_{i} \qquad ( 3 )$$

Step 4: Use the geometric constraints on the end-effector position and orientation. From the previous discussion on the physical structure of the chains and how their first joint axes are oriented in these chains, we have the following constraints on the motion of points on the end-effector.

$$tan 60 ° = \frac{{{ℱ}_{O}}^{} {A}_{2 y}}{{{ℱ}_{O}}^{} {A}_{2 x}} \qquad ( 4 )$$

$$tan 120 ° = \frac{{{ℱ}_{O}}^{} {A}_{3 y}}{{{ℱ}_{O}}^{} {A}_{3 x}} \qquad ( 5 )$$

$${{ℱ}_{O}}^{} {A}_{1 y} = 0 \qquad ( 6 )$$

$${{ℱ}_{O}}^{} {A}_{4 y} = 0 \qquad ( 7 )$$

The above four geometric constraints can be rewritten, where s_{β} represents sin β, c_{β} represents cos β, and r_{A} is the radius of the circle A₁A₂A₃A₄. Detailed expression for [symbol] ₁, [symbol] ₂, [symbol] ₃, [symbol] ₄ are shown in Equations (8) to (11) with θₐ=60°, L, l are predetermined design parameters:

$${{ℱ}_{O}}^{} {A}_{1} = [ \begin{matrix}{{ℱ}_{O}}^{} {P}_{x} \\ {{ℱ}_{O}}^{} {P}_{y} \\ {{ℱ}_{O}}^{} {P}_{z}\end{matrix} ] + {{O}_{}}^{} {R}_{P} [ \begin{matrix}{r}_{A} \\ 0 \\ - ( L - l )\end{matrix} ] = [ \begin{matrix}{{ℱ}_{O}}^{} {P}_{x} + {r}_{A} {c}_{γ} - {c}_{β} {s}_{γ} ( L - l ) \\ {{ℱ}_{O}}^{} {P}_{y} + {s}_{β} ( L - l ) \\ {{ℱ}_{O}}^{} {P}_{z} - {r}_{A} {s}_{γ} - {c}_{β} {c}_{γ} ( L - l )\end{matrix} ] \qquad ( 8 )$$

$${{ℱ}_{O}}^{} {A}_{2} = [ \begin{matrix}{{ℱ}_{O}}^{} {P}_{x} \\ {{ℱ}_{O}}^{} {P}_{y} \\ {{ℱ}_{O}}^{} {P}_{z}\end{matrix} ] {+ \text{}}^{{O}_{}} \text{} {R}_{P} [ \text{} \begin{matrix}{r}_{A} cos {θ}_{a} \\ {r}_{A} sin {θ}_{a} \\ - ( L - l )\end{matrix} ] = [ \begin{matrix}{{ℱ}_{O}}^{} {P}_{x} + {r}_{A} cos {θ}_{a} {c}_{γ} + {r}_{A} sin {θ}_{a} {s}_{β} {s}_{γ} - {c}_{β} {s}_{γ} ( L - l ) \\ {{ℱ}_{O}}^{} {P}_{y} + {r}_{A} sin {θ}_{a} {c}_{β} + {s}_{β} ( L - l ) \\ {{ℱ}_{O}}^{} {P}_{z} - {r}_{A} cos {θ}_{a} {s}_{γ} + {r}_{A} sin {θ}_{a} {s}_{β} {c}_{γ} - {c}_{β} {c}_{γ} ( L - l )\end{matrix} ] \qquad ( 9 )$$

$${{ℱ}_{O}}^{} {A}_{3} = [ \begin{matrix}{{ℱ}_{O}}^{} {P}_{x} \\ {{ℱ}_{O}}^{} {P}_{y} \\ {{ℱ}_{O}}^{} {P}_{z}\end{matrix} ] + {{O}_{}}^{} {R}_{P} [ \begin{matrix}- {r}_{A} cos {θ}_{a} \\ {r}_{A} sin {θ}_{a} \\ - ( L - l )\end{matrix} ] = [ \begin{matrix}{{ℱ}_{O}}^{} {P}_{x} + {r}_{A} cos {θ}_{a} {c}_{γ} + {r}_{A} sin {θ}_{a} {s}_{β} {s}_{γ} - {c}_{β} {s}_{γ} ( L - l ) \\ {{ℱ}_{O}}^{} {P}_{y} + {r}_{A} sin {θ}_{a} {c}_{β} + {s}_{β} ( L - l ) \\ {{ℱ}_{O}}^{} {P}_{z} - {r}_{A} cos {θ}_{a} {s}_{γ} + {r}_{A} sin {θ}_{a} {s}_{β} {c}_{γ} - {c}_{β} {c}_{γ} ( L - l )\end{matrix} ] \qquad ( 10 )$$

$${{ℱ}_{O}}^{} {A}_{4} = [ \begin{matrix}{{ℱ}_{O}}^{} {P}_{x} \\ {{ℱ}_{O}}^{} {P}_{y} \\ {{ℱ}_{O}}^{} {P}_{z}\end{matrix} ] + {{O}_{}}^{} {R}_{P} [ \begin{matrix}- {r}_{A} \\ 0 \\ - ( L - l )\end{matrix} ] = [ \begin{matrix}{{ℱ}_{O}}^{} {P}_{x} + {r}_{A} {c}_{γ} - {c}_{β} {s}_{γ} ( L - l ) \\ {{ℱ}_{O}}^{} {P}_{y} + {s}_{β} ( L - l ) \\ {{ℱ}_{O}}^{} {P}_{z} - {r}_{A} {s}_{γ} - {c}_{β} {c}_{γ} ( L - l )\end{matrix} ] \qquad ( 11 )$$

By combining the constraints equations from (4) to (7) and expressions for [symbol] ᵢ, where i=1 . . . 4, the geometry equation can be written as (12) to (14):

$${}^{{ℱ}_{O}} {P}_{y} = - {s}_{β} ( L - l ) \qquad ( 12 )$$

$$\frac{\sqrt{3} {r}_{A}}{2} {c}_{β} = \sqrt{3} {(}^{{ℱ}_{O}} {P}_{x} + \frac{{r}_{A}}{2} {c}_{γ} + \frac{\sqrt{3} {r}_{A}}{2} {s}_{β} {s}_{γ} - {c}_{β} {s}_{γ} ( L - l ) ) \qquad ( 13 )$$

$$\frac{\sqrt{3} {r}_{A}}{2} {c}_{β} = - \sqrt{3} {(}^{{ℱ}_{O}} {P}_{x} - \frac{{r}_{A}}{2} {c}_{γ} + \frac{\sqrt{3} {r}_{A}}{2} {s}_{β} {s}_{γ} - {c}_{β} {s}_{γ} ( L - l ) ) \qquad ( 14 )$$

On expanding Equations (12), (13), and (14), the lateral bending angle γ and flexion/extension angle β satisfy the relation c_{β}=c_{γ}. There would be 2 solutions, γ=β or γ=−β. The end-effector has the following motion constraints: α=0, γ=β or γ=−β, [symbol] _{y}=−s_{β}(L−l) and [symbol] ₓ=c_{β}s_{γ}(L−l)−\sqrt{3}/2 r_{A}s_{β}s_{γ}. The z-translation of the end-effector [symbol] can be chosen independently. By choosing a pair [ [symbol] γ] with specific values the position and orientation of the end-effector in the base frame can be computed.

Step 5: Given the constraints on the motion of the end-effector position and orientation, the joint variables for each chain SᵢKᵢQᵢEᵢ can be solved using the following equations, where l_{QA}, l_{QE}, l_{KS}, l_{KQ} represent the lengths of QA, QE, KS, and KQ, respectively.

$${‖ {}^{{ℱ}_{O}} {A}_{i} {-}^{{ℱ}_{O}} {Q}_{i} ‖}^{2} = {l}_{QA}^{2} \qquad ( 15 )$$

$${‖ {}^{{ℱ}_{O}} {E}_{i} - {}^{{ℱ}_{O}} {Q}_{i} ‖}^{2} = {l}_{QE}^{2} \qquad ( 16 )$$

Since the computation of joint variables for each chain is similar, we only show the computations for the joint variables θ₁ and ϕ₁ for the chain S₁K₁Q₁E₁. Equations (15) and (16) of the chain S₁K₁Q₁E₁ are written as

$${( {a}_{1} + {v}_{1} )}^{2} + {( {a}_{2} - {v}_{2} )}^{2} = {a}_{3} \qquad ( 17 )$$

$${( {a}_{4} + {v}_{1} )}^{2} + {( {a}_{5} - {v}_{2} )}^{2} = {a}_{6} \qquad ( 18 )$$

where the symbols a₁, . . . , a₆ are determined by the end-effector position and orientation, and the geometric parameters in chain 1. Detailed expressions for aᵢ can be found in the Appendix to this section II. v₁ and v₂ are described below where c₇₄_{₁}_{+ϕ}_{₁} represents cos(θ₁+ϕ₁) and s_{θ}_{₁}_{+ϕ}_{₁} represents sin (θ₁+ϕ₁). To solve the above non-linear Equations (17) and (18), the numerical solver “scipy.optimize.fsolve( )” was used.

$${v}_{1} = {l}_{K S} {c}_{{θ}_{1}} + {l}_{K Q} {c}_{{θ}_{1} + {ϕ}_{1}} \qquad ( 19 )$$

$${v}_{2} = {l}_{K S} {s}_{{θ}_{1}} + {l}_{K Q} {s}_{{θ}_{1} + {ϕ}_{1}} \qquad ( 20 )$$

In summary, one can choose the following two independent variables lateral bending γ, z-translation [symbol] to describe the end-effector. The remaining position and orientation variables x-translation [symbol] , y-translation [symbol] _{y}, flexion/extension β are determined using Step 4. Since each chain SᵢKᵢQᵢ is a planar-two-link mechanism, there will be two solutions for each input pair [symbol] and γ, the “elbow down” and “elbow up” posture. Hence, the mechanism will have 2⁴=16 solutions for each pair of input [ [symbol] , γ] under the condition that γ=β. Similarly, when γ=−β, there should be 16 solutions as well given a pair of input [ [symbol] _{z}, γ]. Thus, for each given [ [symbol] ,γ], the mechanism could have up to 32 solutions, depending on the mechanism parameters.

### 3.4 Forward Kinematics Analysis

The forward kinematics problem is to determine the end-effector position and orientation given the joint rotation angles for motors at S₁ and S₄. As for chain S₁K₁Q₁, the related equations are Equations (21) and (22):

$${a}_{1} {v}_{1} - {a}_{2} {v}_{2} = {a}_{7} - {l}_{K S} {l}_{K Q} {c}_{{ϕ}_{1}} \qquad ( 21 )$$

$${a}_{4} {v}_{1} - {a}_{5} {v}_{2} = {a}_{8} - {l}_{K S} {l}_{K Q} {c}_{{ϕ}_{1}} \qquad ( 22 )$$

As for chain S₄K₄Q₄, the related equations are Equations (23) and (24):

$$- {a}_{1}^{′} {v}_{3} - {a}_{2}^{′} {v}_{4} = {a}_{7}^{′} - {l}_{K S} {l}_{K Q} {c}_{{ϕ}_{4}} \qquad ( 23 )$$

$$- {a}_{4}^{′} {v}_{3} - {a}_{5}^{′} {v}_{4} = {a}_{8}^{′} - {l}_{K S} {l}_{K Q} {c}_{{ϕ}_{4}} \qquad ( 24 )$$

Where v₃ and v₄ are expressed in Equations (25) and (26), which are similar to v₁ and v₂.

$${v}_{3} = {l}_{K S} {c}_{{θ}_{4}} + {l}_{K Q} {c}_{{θ}_{4} + {ϕ}_{4}} \qquad ( 25 )$$

$${v}_{4} = {l}_{K S} {s}_{{θ}_{4}} + {l}_{K Q} {s}_{{θ}_{4} + {ϕ}_{4}} \qquad ( 26 )$$

aᵢ symbols and aᵢ′ symbols in Equations (21) to (24) are determined by end-effector position and orientation, and the design parameters. Detailed expressions for aᵢ and aᵢ′ are written in the Appendix.

In conclusion, Equations (21) and (22) can be written as Equations (27) and (28). In addition, Equations (23) and (24) can be written as Equations (29) and (30).

$${f}_{1} ( {θ}_{1} , {ϕ}_{1} , {}^{{ℱ}_{O}} {P}_{x} {,}^{{ℱ}_{O}} {P}_{y} {,}^{{ℱ}_{O}} {P}_{z} , β , γ ) = 0 \qquad ( 27 )$$

$${f}_{2} ( {θ}_{1} , {ϕ}_{1} , {}^{{ℱ}_{O}} {P}_{x} {,}^{{ℱ}_{O}} {P}_{y} {,}^{{ℱ}_{O}} {P}_{z} , β , γ ) = 0 \qquad ( 28 )$$

$${f}_{3} ( {θ}_{4} , {ϕ}_{4} , {}^{{ℱ}_{O}} {P}_{x} {,}^{{ℱ}_{O}} {P}_{y} {,}^{{ℱ}_{O}} {P}_{z} , β , γ ) = 0 \qquad ( 29 )$$

$${f}_{4} ( {θ}_{4} , {ϕ}_{4} , {}^{{ℱ}_{O}} {P}_{x} {,}^{{ℱ}_{O}} {P}_{y} {,}^{{ℱ}_{O}} {P}_{z} , β , γ ) = 0 \qquad ( 30 )$$

Since in the forward kinematics, [symbol] ₓ, [symbol] _{y}, β are determined by [symbol] _{z}, γ, the unknown variables are [symbol] _{z}, γ, ϕ₁, and ϕ₄. The provided input pair now are joint variables θ₁ of the motor placed on S₁ and joint variable θ₄ of the motor placed on S₄. Therefore, there are 4 equations related to 4 unknown variables. For solving numerically, initial guesses were provided for the four variables to solve the forward kinematics problem. For each pair of [θ₁, θ₄], we expect the mechanism to have 8 solutions.

### 3.5 Simulation of Kinematics in SolidWorks and Python

To verify the correctness of inverse and forward kinematics, we used SolidWorks Model and Python code.

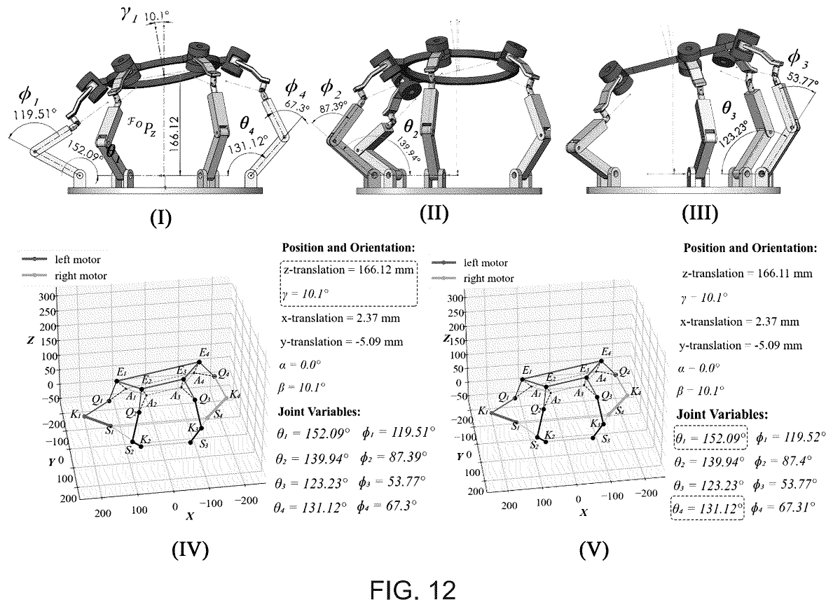

In SolidWorks, we built a model for the 4-chain-RRUR mechanism. With this model, we measured the end-effector position/orientation and the joint variables. Additionally, we coded the equations listed in Section 3.3 to create a Python model with the same set of design parameters as used in SolidWorks. For Inverse Kinematics (IK), we provided several pairs of [ [symbol] , γ] to the IK model. The IK model in Python outputs the joint variables. One case with inputs of [symbol] =166.12 mm and γ=10.1° is shown in FIG. 12.

FIGS. 12-(I), 12-(II), and 12-(III) show three views of the SolidWorks model. Measurements of θ₁ and ϕ₁ are shown in FIG. 12-(I), θ₂ and ϕ₂ in FIG. 12-(II), and θ₃ and ϕ₃ in FIG. 12-(III). Moreover, the results from Python with the input position [symbol] =166.12 mm and γ=10.1° are shown in FIG. 12-(IV), the dashed rectangle in FIG. 12-(IV) shows the input parameters to the inverse kinematics solver. Notably, the results from python model exactly match the results measured in SolidWorks. This is a validation of the inverse kinematics in Section 3.3.

For forward kinematics, the input variables are θ₁ and θ₄. For each given pair [θ₁, θ₄], posture of chain S₁K₁Q₁ and chain S₄K₄Q₄ are then determined. As stated in Section 3.4, for each given pair [θ₁, θ₄] with the condition θ₁≠θ₄, we expect 8 solutions (4 under γ=β and 4 under γ=−β). Here, we directly used the results of θ₁ and θ₄ from the inverse kinematics shown in FIG. 12-(IV) and sent these to forward kinematics solver. Results are shown in FIG. 12-(V) with the input pair [152.09°, 131.12° ] marked by dashed rectangle. The output posture of the forward kinematics in FIG. 12-(V) matches the posture of the inverse kinematics within a small computation error. This is a numerical validation of the kinematics in Section 3.3 and 3.4.

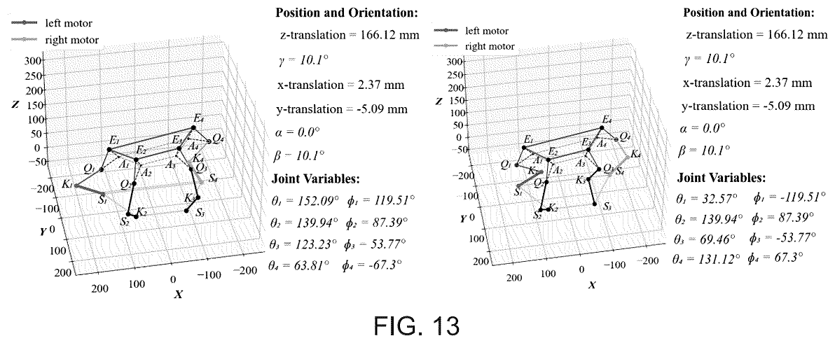

Since the neck brace will be attached on the human within the head-neck area, the chain SᵢKᵢQᵢ will be unsafe due to potential interference with the human neck if the θᵢ<90° (as shown in FIG. 13). Thus, the neck brace should be assembled and operated in the mode θᵢ>90°.

### 3.6 Workspace Comparison

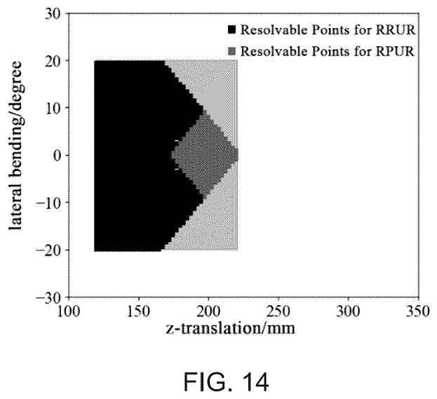

We plotted the workspace with the horizontal axis as the z-translation [symbol] and the vertical axis as the lateral bending angle γ. If a point in this 2D workspace of [ [symbol] , γ] is reachable when computing the inverse kinematics, we mark this pair as reachable. The workspaces for both the 4-chain-RPUR and 4-chain-RRUR mechanisms are shown in FIG. 14. Both these mechanisms have the same end-effector constraints on the motion. Since one would benefit by a larger workspace of the end-effector, we compared its workspace with that of a 4-chain-RPUR mechanism. Both designs have the same 2DOF of the end-effector. As discussed above in connection with FIG. 9 the main difference is that the second joint is changed from a prismatic joint to a revolute joint, while keeping the other structures within the chains.

In FIG. 14, we chose the limits of the workspace to be lateral bending γ=[−20°, 20°] and z-translation [symbol] =[120 mm, 221 mm]. The workspace of 4-chain-RPUR mechanism has a diamond shape shown in blue. The workspace of 4-chain-RRUR mechanism has a pentagon shape shown in black which includes the workspace obtained with the 4-chain-RPUR. These workspaces do not account for any geometric collision between the links of the chains or with the human head.

### 4. Validation of the Prototype

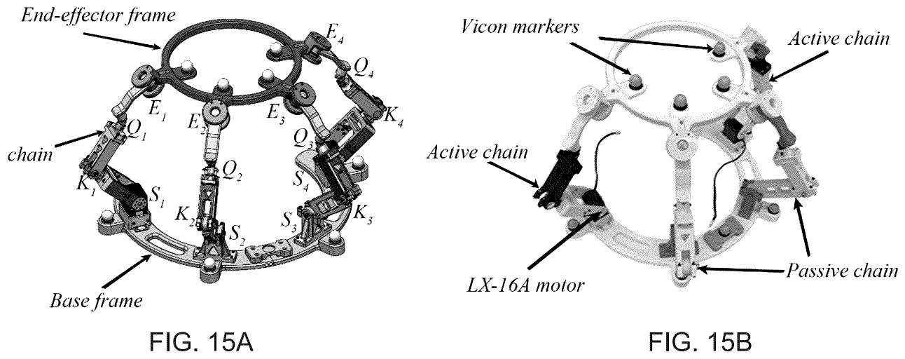

We fabricated a benchtop prototype of the FIG. 10 embodiment and used LX-16A bus servos as motors to activate the mechanism. The SolidWorks model and fabricated prototype are depicted in FIGS. 15A and 15B, respectively. The prototype was built to test the motion of the mechanism using a Vicon Nexus motion capture system. We used a flat platform base. FIG. 15B shows the Vicon markers placed on the prototype. The important dimensions of the prototype are shown in Table 2.

TABLE 2
Key dimensions of the fabricated prototype (Unit: mm)

| E₁E₄ | S₁S₄ | QᵢKᵢ | QᵢEᵢ |
|---|---|---|---|
| 240 | 282 | 83.925 | 65.36 |

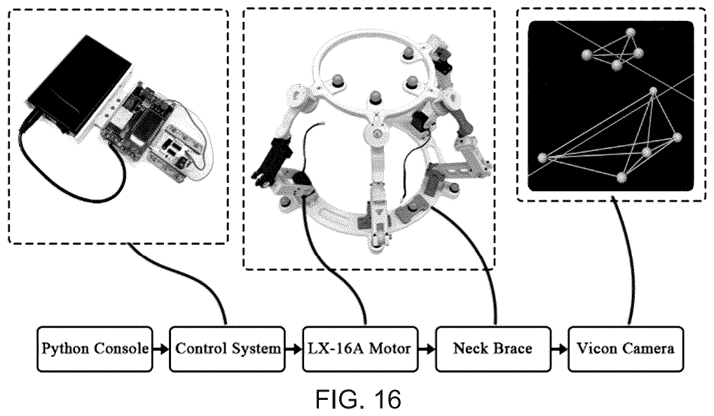

In the prototype, the control system was composed of a battery with 12V output, a DC converter, and a bus linker controller. The DC converter was used to convert the battery output of 12V to the bus servo input of 7V. The servo motors were connected to the ports of the controller. Before validation, Vicon markers were attached to points on the component rigid bodies to measure the locations of E₁, . . . , E₄ and S₁, . . . , S₄. Extra Vicon markers were placed on the rigid bodies to prevent the cameras from losing images of Vicon markers. In our current validation experiment, an extra Vicon marker was placed on the base frame. The workflow of the validation experiment is depicted in FIG. 16.

For discrete position control, a pair [ [symbol] , γ] is sent to motors by the Python console. The joint variables of two rotary motors are computed by the inverse kinematics algorithm. These commands are sent to the motors and Vicon markers on the end-effector and base frame are recorded. From these data, the z-translation and lateral bending values of the end-effector are calculated.

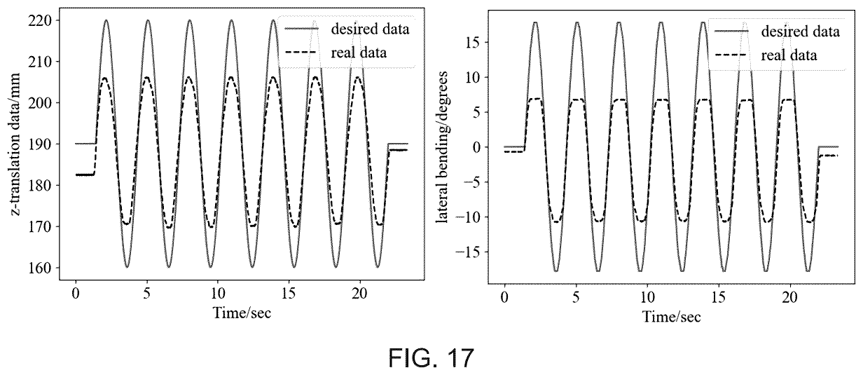

For the continuous motion of the end-effector, sequences of pure z-translation and z-translation with lateral bending were sent as commands for the motors. The results of the desired motion sequence and captured motion sequence were processed with Vicon Nexus software and are shown in FIG. 17. More specifically, FIG. 17 shows the plots of the measured z-translation and lateral bending angles along with the commanded values. The red lines are the commanded data to the mathematical model whereas the black dashed lines are the measured data. The errors between the desired commands and the measurement from Vicon are presented in Table 3.

TABLE 3
Position control errors

| | Lateral bending/° | Z-translation/mm |
|---|---|---|
| MEAN Errors | 5.30 | 7.04 |
| RMS Errors | 5.93 | 8.12 |

Potential Errors in Fabrication and Testing: We have made the assumption that the points A₁ and A₄ stay in the same plane fixed to the base frame in all configurations. This assumption may not be fully met due to fabrication errors and link flexibility of 3D-printed parts used to construct the mechanism. Several related works have studied how to reduce position errors within a design. In these studies, error models were made to identify the most crucial geometric error and develop a method to control this error and improve the performance of position control.

In the fabricated mechanism, there are also potential interference of the links, and as a result, the motion of the end-effector can be smaller than the theoretically computed range. Furthermore, it was hard to measure the passive angles in the universal joints in the prototype. Hence, the errors in the universal joints could be determined. Despite these errors, the continuous motion of the end-effector shows a close match between the motion of the physical prototype and the mathematical kinematics model.

### 5. Conclusion

Section II describes a 4-RRUR parallel mechanism that provides 2 DOF motion to the end-effector. The vertical z-translation of the end-effector can be used to provide traction on the human head while the orientation DOF can be applied to orient the head appropriately during traction. We characterized the inverse and forward kinematics of this parallel mechanism in this section. The workspace of this 4-RRUR parallel mechanism is larger in both lateral bending and vertical z-translation compared to 4-RPUR parallel mechanisms. The prototype is validated using VICON as the end-effector performed lateral bending and vertical z-translation. Overall, the fabricated prototype mimics the mathematical model well, despite potential errors in fabrication. This design opens up new possibilities for its use in medical applications to relieve neck pain.

### 6. Appendix for Section II

The following Equations are the expressions for aᵢ and aᵢ′ symbols mentioned above in section II-3.3. r_{E} is the radius of the end-effector headband circle, r_{S} represents the radius of the base frame circle, r_{A} represents the radius of the intermediate frame circle formed by A₁, A₂, A₃, A₄.

$${a}_{1} {}^{{ℱ}_{O}} {P}_{x} + {c}_{γ} {r}_{E} - {r}_{S} \qquad ( 31 )$$

$${a}_{2} {}^{{ℱ}_{O}} {P}_{z} - {s}_{γ} {r}_{E} \qquad ( 32 )$$

$${a}_{3} = {l}_{QE}^{2} {-}^{{ℱ}_{O}} {P}_{y}^{2} \qquad ( 33 )$$

$${a}_{4} {=}^{{ℱ}_{O}} {P}_{x} + {c}_{γ} {r}_{A} - {c}_{β} {s}_{γ} ( L - l ) - {r}_{S} \qquad ( 34 )$$

$${a}_{5} {=}^{{ℱ}_{O}} {P}_{z} - {s}_{γ} {r}_{A} - {c}_{β} {c}_{γ} ( L - l ) \qquad ( 35 )$$

$${ {a}_{6} = {l}_{Q A}^{2} - {(}^{{ℱ}_{O}} {P}_{y} + ( L - l ) {s}_{β} )}^{2} \qquad ( 36 )$$

$${a}_{7} = \frac{{a}_{3} - {a}_{1}^{2} - {a}_{2}^{2} - {l}_{K S}^{2} - {l}_{K Q}^{2}}{2} \qquad ( 37 )$$

$${a}_{8} = \frac{{a}_{6} - {a}_{4}^{2} - {a}_{5}^{2} - {l}_{K S}^{2} - {l}_{K Q}^{2}}{2} \qquad ( 38 )$$

$${a}_{1}^{′} {=}^{{ℱ}_{O}} {P}_{x} - {c}_{γ} {r}_{E} + {r}_{S} \qquad ( 39 )$$

$${a}_{2}^{′} {=}^{{ℱ}_{O}} {P}_{z} + {s}_{γ} {r}_{E} \qquad ( 40 )$$

$${a}_{3}^{′} = {l}_{QE}^{2} {-}^{{ℱ}_{O}} {P}_{y}^{2} \qquad ( 41 )$$

$${a}_{4}^{′} {=}^{{ℱ}_{O}} {P}_{x} - {c}_{γ} {r}_{A} - {c}_{β} {s}_{γ} ( L - l ) + {r}_{S} \qquad ( 42 )$$

$${a}_{5}^{′} {=}^{{ℱ}_{O}} {P}_{z} + {s}_{γ} {r}_{A} - {c}_{β} {c}_{γ} ( L - l ) \qquad ( 43 )$$

$${{a}_{6}^{′} = {l}_{Q A}^{2} - {(}^{{ℱ}_{O}} {P}_{y} + ( L - l ) {S}_{β} )}^{2} \qquad ( 44 )$$

$${a}_{7}^{′} = \frac{{a}_{3}^{′} - {a}_{1}^{′2} - {a}_{2}^{′2} - {l}_{K S}^{2} - {l}_{K Q}^{2}}{2} \qquad ( 45 )$$

$${a}_{8}^{′} = \frac{{a}_{6}^{′} - {a}_{4}^{′2} - {a}_{5}^{′2} - {l}_{K S}^{2} - {l}_{K Q}^{2}}{2} \qquad ( 46 )$$

### Section III

### 1. Introduction

Existing manual and mechanical methods of applying traction to the head-neck are limited due to variability in the applied forces and the orientation of the head-neck relative to the shoulders during the procedure. Existing robotic neck braces are not designed to provide independent rotation angles and independent vertical translation, or traction, to the brace end-effector connected to the head, making them unsuitable for traction application. This section III describes a robotic neck brace that can provide vertical traction to the head while keeping the head in a prescribed orientation, with flexion and lateral bending angles. In this section, the kinematics of the end-effector attached to the head relative to a coordinate frame on the shoulders are described as well as the velocity kinematics and force control.

This section also describes benchtop experiments designed to validate the position control and the ability of the brace to provide a vertical traction force. For one embodiment, the maximum achievable end-effector orientations were 16° in flexion, 13.9° in extension, and ±6.5° in lateral bending. The kinematic model of the active brace was validated using an independent motion capture system with a maximum root mean square error of 2.41°. In three different orientations of the end-effector, neutral, flexed, and laterally bent, the brace was able to provide a consistent upward traction force during intermittent force application. In these configurations, the force error has standard deviations of 0.55, 0.29, and 0.07 N, respectively. This section validates the mechanism's ability to achieve a range of head orientations and provide consistent upward traction force in these orientations, making it a suitable intervention tool in cases of cervical disk degeneration.

Existing methods of applying traction have limitations either due to human variability or physical inability to provide traction to a patient in a lateral bending orientation. Existing Robotic neck braces, while allowing both flexion-extension and lateral bending, cannot independently control the vertical translation.

The ability to provide traction force in a lateral bending position has been shown to improve cervical range of motion and reduce neck pain. But heretofore, no mechanical traction devices could keep the head in a laterally bent position or provide control over the angular orientation of the head during traction while controlling the traction force.

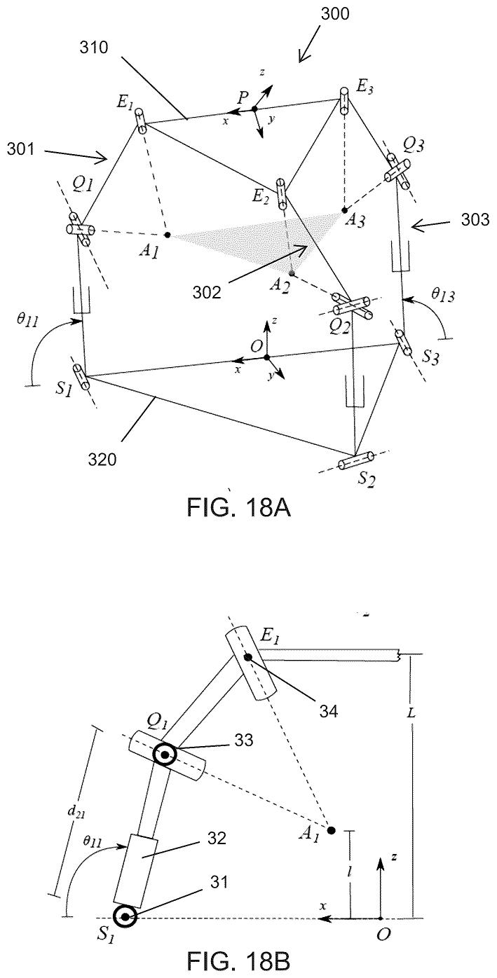

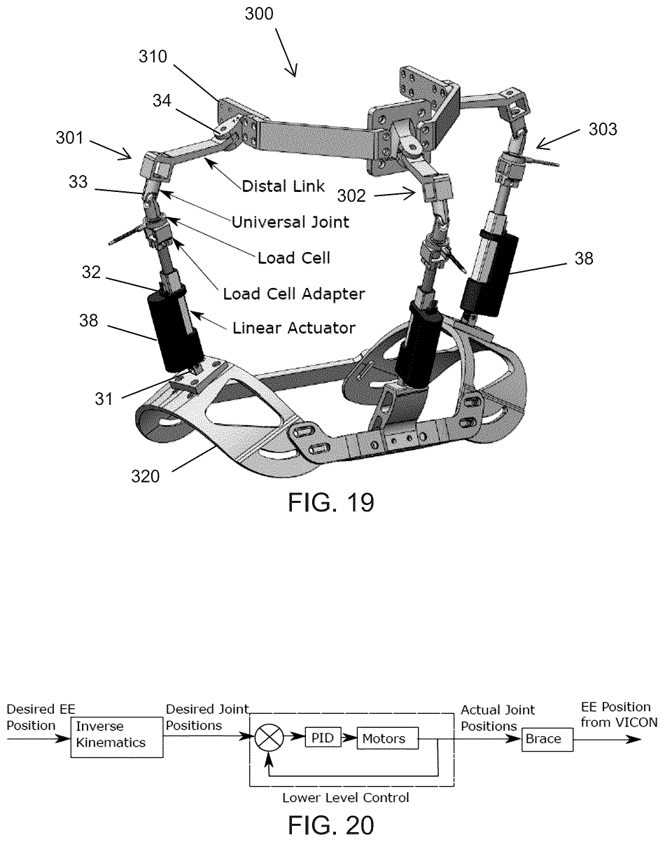

FIGS. 18-19 depict a neck brace 300 that addresses these deficiencies. More specifically, FIG. 18A is a schematic diagram of the neck brace 300, with the base frame defined by the shoulders, with an origin at point O. The end-effector frame is at the head and has an origin at point P. FIG. 18B is a schematic diagram of one chain of the FIG. 18A neck brace. And FIG. 19 depicts a CAD of the neck brace 300 with the parts within one of the chains 301 labeled. FIG. 19 also shows the load cells and linear actuators in each chain.

The neck brace 300 has a base frame 320 that is configured to be supported by the subject's shoulder, an end-effector frame 310 configured so that it can be removably attached to the subject's head (e.g., using a headband positioned at the subject's forehead and/or neck straps), and three chains of joints 301, 302, 303. Each of these chains of joints runs between the base frame 320 and the end-effector 310. S1, S2, and S3 are on the base frame 320; and E1, E2, and E3 are attached to the end-effector 310 at the head. Each of the three chains 301, 302, 303 has an RPUR structure (which means that, beginning at the base frame 320 and moving up towards the end-effector 310, the order of the joints is: a lower revolute joint 31 that is connected to the base frame 320, a prismatic joint 32, a universal joint 33, and an upper revolute joint 34). The upper revolute joint 34 is connected to the end-effector frame 310.

The embodiment depicted in FIGS. 18-19 also includes a plurality of linear actuators 38, each of which is configured to actively control a respective one of the prismatic joints 32. For example, as depicted in FIG. 19, each of the three linear actuators 38 is configured to actively control a respective one of the three prismatic joints 32. In alternative embodiments (not shown) only the two outer linear actuators 38 are configured to actively control the two lateralmost prismatic joints 32. Any of the prismatic joints 32 that are not actively controlled by one of the linear actuators 38 are passive prismatic joints. Optionally, at least one additional chain of joints (not shown) having a first end connected to the base frame 320 and a second end connected to the end-effector frame 310 can be added. The universal joint 33 can be implemented using two revolute joints.

Due to the specific architecture of the individual chains, it can be verified, using the principles of screw theory, that the end-effector 310 has an independent vertical translation in the inertial frame aligned with the shoulders. Notably, the neck brace 300 can apply an independent vertical translation to the end-effector 310, along with degrees-of-freedom of the end-effector 310 in both flexion-extension and lateral bending. A prototype neck brace 300 was designed, constructed, and validated to demonstrate these features in this section.

### 2. Mathematical Model

### A. Mechanism Architecture

The neck brace 300 has three RPUR chains 301-303, and the end-effector 310 has three degrees-of-freedom. This structure provides flexion, extension, lateral bending, and vertical translation roughly parallel to the cervical spine relative to the shoulders. A three-chain mechanism was selected to be placed around the head and shoulders in a wearable application.

Each chain has the same RPUR structure. The chains are placed such that the axes of the lower revolute joints (Sᵢ) for chains 301 and 303 are parallel to each other and perpendicular to the axis of the lower revolute joint of chain 302, as best seen in FIG. 18. The universal joint consists of two revolute joints, whose axes intersect at the point Qᵢ. The axis of the second revolute joint on the universal joint and the axis of the final revolute joint on the chain intersect at the points Aᵢ. The prismatic joints between Sᵢ and Qᵢ are the active joints in this design.

FIG. 19 is a CAD model of the neck brace 300 where chains 301 and 303 sit on the left and right shoulders, respectively, while chain 302 is attached behind the neck, taking support from the shoulders. The end-effector 310 is configured to interface with the upper half of the subject's head (e.g., using headband and/or neck straps).

Examination of the constraints on key points of the mechanism reveals that A₁ and A₃ are constrained to the xz plane of the base frame 320. Therefore, the plane created by Aᵢ cannot rotate about the z-axis. Since the plane Aᵢ is on the rigid body of the end-effector 310, it can be concluded that the end-effector 310 cannot rotate about the z-axis and the rotation in the horizontal plane (α) must be zero.

### B. Inverse Kinematics

The prismatic joint lengths in chains 301, 302, and 303 (d₂₁, d₂₂, d₂₃) were computed given a desired end-effector flexion angle (β), lateral bending angle (γ), and vertical position (^{F}^{_{O}}P_{z}). The rotation matrix ^{O}R_{P} of the base frame, F_{O}, to the end effector frame, F_{P}, was described as a Space 3-1-2 rotation with angles [α, β, γ].

$${}^{O} {R}_{P} = [ \begin{matrix}s α s β s γ + c γ & s β s γ - c γ s α & c β s γ \\ s α c β & c α c β & - s β \\ s α s β c γ - s γ & c α s β c γ + s γ s α & c β c γ\end{matrix} ]$$

where s and c are abbreviations for sin and cos, respectively.

As previously established, α=0, which allows the rotation matrix to be simplified further.

$${}^{O} {R}_{P} = [ \begin{matrix}c γ & s β s γ & c β s γ \\ 0 & c β & - s β \\ - s γ & s β c γ & c β c γ\end{matrix} ]$$

The locations of Aᵢ and Eᵢ in the base frame were computed using the rotation matrix from the base frame to the end-effector frame, ^{O}R_{P}, where i refers to the chain being analyzed.

$${}^{{ℱ}_{O}} {A}_{i} {=}^{{ℱ}_{O}} P + {}^{O} {R}_{P} {}^{{ℱ}_{P}} {A}_{i} , i = 1 , 2 , 3 \qquad ( 1 )$$

$${}^{{ℱ}_{O}} {E}_{i} {=}^{{ℱ}_{O}} P + {}^{O} {R}_{P} {}^{{ℱ}_{P}} {E}_{i} , i = 1 , 2 , 3 \qquad ( 2 )$$

Due to the constraints on the y positions of A₁ and A₃, as well as the constraint on the x position of A₂, a relationship was computed between the angles β, γ, and the Cartesian positions of the end-effector. By setting the expressions for A_{1y} and A₂ₓ equal to zero, the relationship between these values was found and is displayed in (3). For simplicity, the position of the origin on the end-effector in the base frame will be written as x, y, and z, moving forward in this section.

$$y = - {}^{{ℱ}_{P}} {A}_{1 x} c β - s β ( L - l ) \qquad ( 3 )$$

$$x = - {}^{{ℱ}_{P}} {A}_{2 y} s β s γ + c β s β ( L - l )$$

The lengths ∥ EᵢQᵢ∥ and ∥AᵢQᵢ∥ are constants and known. Using these lengths, two equations were created for each chain i.

$${{‖}^{{F}_{o}} {A}_{i} {-}^{{F}_{o}} {Q}_{i} ‖}^{2} = {‖ {A}_{i} {Q}_{i} ‖}^{2} \qquad ( 4 )$$

$${‖ {F}_{o} {E}_{i} {-}^{{F}_{o}} Q ‖}^{2} = {‖ {E}_{i} {Q}_{i} ‖}^{2} \qquad ( 5 )$$

$$i = 1 , 2 , 3$$

Equations (4) and (5) are simplified and recombined into (6) and (7), The values of a₁ᵢ, a₂ᵢ, a₃ᵢ, a₄ᵢ, a₅ᵢ, and a₆ᵢ are known given the end-effector orientations [β, γ, z].

$${( {a}_{1 i} − {b}_{1 i} )}^{2} + {( {a}_{2 i} − {b}_{2 i} )}^{2} = {a}_{3 i} \qquad ( 6 )$$

$${( {a}_{4 i} − {b}_{1 i} )}^{2} + {( a \text{?} − {b}_{2 i} )}^{2} = {a}_{6 i} \qquad ( 7 )$$

$$where ,$$

$${b}_{1 i} = {d}_{2 i} sin {θ}_{1 i}$$

$${b}_{2 i} = {d}_{2} \text{?} cos {θ}_{1} \text{?}$$

$$\text{?} \text{indicates text missing or illegible when filed}$$

Using the trigonometric identity s²θ₁ᵢ+c²θ₁ᵢ=1, (6) and (7) were simplified and combined to create a matrix equation, where a₇ᵢ and a₈ᵢ are functions of a₁ᵢ, a₂ᵢ, a₃ᵢ, a₄ᵢ, a₅ᵢ, a₆ᵢ, and d₂ᵢ.

$$[ \begin{matrix}{a}_{1 i} & {a}_{2 i} \\ {a}_{4 i} & a \text{?}\end{matrix} ] [ \begin{matrix}s {θ}_{1 i} \\ s {θ}_{1 i}\end{matrix} ] = [ \begin{matrix}{a}_{7 i} \\ {a}_{8 i}\end{matrix} ] \qquad ( 8 )$$

$$\text{?} \text{indicates text missing or illegible when filed}$$

After solving for sθ₁ᵢ and cθ₁ᵢ, the trigonometric identity s²θ₁ᵢ+c²θ₁ᵢ=1 was used again to yield the following scalar equation.

$$\begin{matrix}( & {{a}_{5 i} {a}_{7 i} - {a}_{2 i} a \text{?} )}^{2}\end{matrix} + {( {a}_{4 i} {a}_{7 i} - {a}_{1 i} {a}_{8 i} )}^{2} = ( {{α}_{1 i} {a}_{5 i} - {a}_{2 i} {a}_{4 i} )}^{2} \qquad ( 9 )$$

$$\text{?} \text{indicates text missing or illegible when filed}$$

Equation (9) can be solved numerically for each chain to determine the joint lengths [d₂₁, d₂₂, d₂₃] for a commanded β, γ, and z.

### C. Forward Kinematics

The forward kinematics of this system is defined as finding the flexion angle, β, the lateral bending angle γ, and the z position of the end-effector, z, given the prismatic joint lengths in chains 301, 302, and 303, i.e., d₂₁, d₂₂, d₂₃. This is accomplished by finding (9) for each chain. This set of three equations relates the three unknown variables, [β, γ, z], to the known variables d₂₁, d₂₂, d₂₃. All other variables in the equations are known based on the geometry of the brace 300.

This system of equations can be solved numerically. In order to reduce error due to multiple solutions, an initial guess for the values of [β, γ, z] is selected. During real-time computation, the initial guess is set as the [β, γ, z] values of the brace from the previous time step.

### D. Jacobian

During force control, the forces of interest are the three Cartesian forces: Fₓ, F_{y}, and F_{z}. Therefore, (6) and (7) were modified such that they were in terms of d₂ᵢ, θ₁ᵢ, x, y, and z. The relationships between β, γ, x, and y were previously derived in (3). These equations were then differentiated with respect to time, yielding the following three sets of two equations.

$${A}_{1 i} {\dot{d}}_{2 i} + {B}_{1 i} {\dot{θ}}_{1 i} + {C}_{1 i} \dot{x} + {D}_{1 i} \dot{y} + {E}_{1 i} \dot{z} = 0 \qquad ( 10 )$$

$${A}_{2 i} {\dot{d}}_{2 i} + {B}_{2 i} {\dot{θ}}_{1 i} + {C}_{2 i} \dot{x} + {D}_{1 i} \dot{y} + {E}_{2 i} \dot{z} = 0 \qquad ( 11 )$$

$$i = 1 , 2 , 3$$

where the coefficients A₁ᵢ, A₂ᵢ, B₁ᵢ, B₂ᵢ, C₁ᵢ, C₂ᵢ, D₁ᵢ, D₂ᵢ, E₁ᵢ, E₂ᵢ are dependent on the brace configuration.

Equation (10) was solved for θ′1i and substituted into (11). This results in 3 equations which can be combined to create the matrix equation (12) which relates the joint velocities to the Cartesian end-effector velocities.

$$H [ \begin{matrix}{\dot{d}}_{21} \\ {\dot{d}}_{22} \\ {\dot{d}}_{23}\end{matrix} ] = K [ \begin{matrix}\dot{x} \\ \dot{y} \\ \dot{x}\end{matrix} ] \qquad ( 12 )$$

$$where$$

$$H = [ \begin{matrix}{A}_{22} − \frac{A \text{?} B \text{?}}{{B}_{11}} & 0 & 0 \\ 0 & {A}_{22} − \frac{A \text{?} {B}_{22}}{{B}_{12}} & 0 \\ 0 & 0 & {A}_{23} − \frac{A \text{?} B \text{?}}{{B}_{13}}\end{matrix} ]$$

$$K = [ \begin{matrix}{C}_{21} - \frac{{B}_{21} {C}_{11}}{{B}_{11}} & {D}_{21} - \frac{{B}_{21} {D}_{11}}{{B}_{11}} & {E}_{21} - \frac{{B}_{21} {E}_{11}}{{B}_{11}} \\ {C}_{22} - \frac{{B}_{22} {C}_{12}}{{B}_{12}} & {D}_{22} - \frac{{B}_{22} {D}_{12}}{{B}_{12}} & {E}_{22} - \frac{{B}_{21} {E}_{12}}{{B}_{12}} \\ {C}_{22} - \frac{{B}_{23} {C}_{13}}{{B}_{13}} & {D}_{23} - \frac{{B}_{23} {D}_{13}}{{B}_{13}} & {E}_{22} - \frac{{B}_{21} {E}_{13}}{{B}_{13}}\end{matrix} ]$$

$$\text{?} \text{indicates text missing or illegible when filed}$$

Therefore, the velocity kinematics of the mechanism can be described as follows.

$$[ \begin{matrix}{ω}_{1} \\ {ω}_{2} \\ {ω}_{2}\end{matrix} ] = J [ \begin{matrix}{\dot{d}}_{21} \\ {\dot{d}}_{22} \\ {\dot{d}}_{23}\end{matrix} ] \qquad ( 13 )$$

where J=K⁻¹H.

Simulation analysis confirms that the matrix K remains invertible throughout the range of motion of the mechanism.

### E. Force Control

Due to the constraints on the degrees-of-freedom of the mechanism, there is a relationship between select Cartesian velocities and angular velocities of the end-effector. The relationships between the measure numbers of angular velocity of the end-effector and the rate of change of the Euler angles in a Space 3-1-2 rotation can be determined. As α=0, the equations can be further simplified.

$${ω}_{1} = \dot{β} \qquad ( 14 )$$

$${ω}_{2} = \dot{γ} \text{?}$$

$$ω \text{?} = \dot{γ} {s}_{β}$$

$$\text{?} \text{indicates text missing or illegible when filed}$$

where ωᵢ are the measure numbers of the angular velocity of the end-effector along the coordinate directions of the end-effector. The time derivative of the position constraint equations in (3) can be computed using a symbolic toolbox, yielding the following equations, where the coefficients f, g, h are dependent on brace geometry and configuration.

$$\dot{x} = f \dot{β} + g \dot{γ} \qquad ( 15 )$$

$$\dot{y} = h \dot{β}$$

These equations can be solved for β′ and γ′, which can then be substituted into (14), resulting in three equations that relate the components of the angular velocity and Cartesian velocity of the end-effector. These equations can then be put into matrix form, yielding the velocity constraint matrix G.

$$[ \begin{matrix}{ω}_{1} \\ {ω}_{2} \\ {ω}_{2}\end{matrix} ] = {[ \begin{matrix}0 & \frac{1}{\text{?}} & 0 \\ {c}_{β} & − \frac{\text{?}}{\text{?}} & 0 \\ \frac{\text{?}}{g} & \frac{\text{?}}{{g}_{h}} & 0\end{matrix} ]}_{\underbrace{G}} [ \begin{matrix}\dot{x} \\ \dot{y} \\ \dot{z}\end{matrix} ] \qquad ( 16 )$$

$$\text{?} \text{indicates text missing or illegible when filed}$$

The joint actuator forces [F₁, F₂, F₃] can be related to the end-effector forces and moments by equating the rate of work done by the actuators to the rate of work done on the environment by the end-effector.

$$[ \begin{matrix}\begin{matrix}{F}_{1} & {F}_{2}\end{matrix} & {F}_{3}\end{matrix} ] \dot{q} = {F}^{T} \dot{X} \qquad ( 17 ) ]$$

where F^{T}=[Fₓ, F_{y}, F_{z}, Mₓ, M_{y}, M_{z}], the end-effector forces exerted on the environment, X′=[x′, y′, z′, ω1, ω2, ω3], and q′=[d₂₁, d₂₂, d₂₃].

On substituting (16) into (17) and collecting terms, we get the following force relationship:

$$[ \begin{matrix}\begin{matrix}{F}_{1} & {F}_{2}\end{matrix} & {F}_{3}\end{matrix} ] = ( [ \begin{matrix}\begin{matrix}{F}_{x} & {F}_{y}\end{matrix} & {F}_{z}\end{matrix} ] + [ \begin{matrix}\begin{matrix}{M}_{x} & {M}_{y}\end{matrix} & {M}_{z}\end{matrix} ] G ) J \qquad ( 18 )$$

It is important to observe the structure of (18). As the third column of G consists of all zeros, the third element of ([Fₓ F_{y} F_{z}]+[Mₓ M_{y} M_{z}]G) is F_{z}, the vertical force along the z direction. This feature allows for independent control of vertical traction force.

### 3. Methods

### A. Prototype

The brace 300 is primarily constructed of 3-D printed parts, along with off-the-shelf parts for fasteners, joint hardware, motors, and force sensors. The lower revolute joint of each chain uses the existing joint axis at the bottom of the linear actuator at Sᵢ, as best seen in FIG. 19. A custom 3-D printed adapter was designed to attach one side of a Futek LCM 200 load cell to the end of the linear actuator. Connected to the other side of the load cell is a universal joint, which contains two revolute joints, and is located at the intersection points of the revolute joint axes, at point Qᵢ. Finally, the distal link is attached to the end of the universal joint and at point Eᵢ, the chain attaches to the end-effector 310. The Actuonix P16-100-64-12-P linear actuators that were used in the prototype use a dc motor with an integrated encoder to deliver position feedback to the microcontroller. The load cells are connected to Futek amplifiers, which allow the microcontroller to read the forces. This brace 300 utilizes a Photon microcontroller for low-level control of the motors in both position and force control modes. The high-level control is conducted on a desktop computer which streams position or force commands to the microcontroller over Wi-Fi. Of course, in alternative embodiments, components other than the ones listed in this paragraph can be used.

### B. Position Control

A benchtop experiment was designed to validate position control of the brace 300. Commands for end-effector orientation and position were sent in a sinusoidal pattern ranging from the minimum to maximum values of flexion-extension movement, lateral bending, and vertical translation. Based on the mathematical model, taking into consideration the stroke length of the linear actuators, the maximum achievable end-effector angles in flexion-extension motion are 21.1° and −16.6°, where the positive and negative values refer to the flexion and extension angles, respectively. The maximum lateral bending angle is 8.25° symmetrically in each direction. In the neutral configuration, the vertical range of motion is 50 mm. The brace was commanded to complete 3 cycles of motion in each direction with a period of 25 s.

The linear actuator lengths were computed from the desired position and orientation and were sent to the microcontroller at 50 Hz, following the position control block diagram depicted in FIG. 20. A VICON motion capture system was used to measure the achieved position and orientation of the end-effector 310 and the base frame 320. Three retroreflective markers were placed near points Eᵢ, and three near points Sᵢ in order to define the end-effector and base frames, respectively. The commanded and achieved joint positions were recorded along with the orientation and position of the end-effector 310 relative to the base frame 320 by using a VICON Lock Lab control box.

### C. Force Control

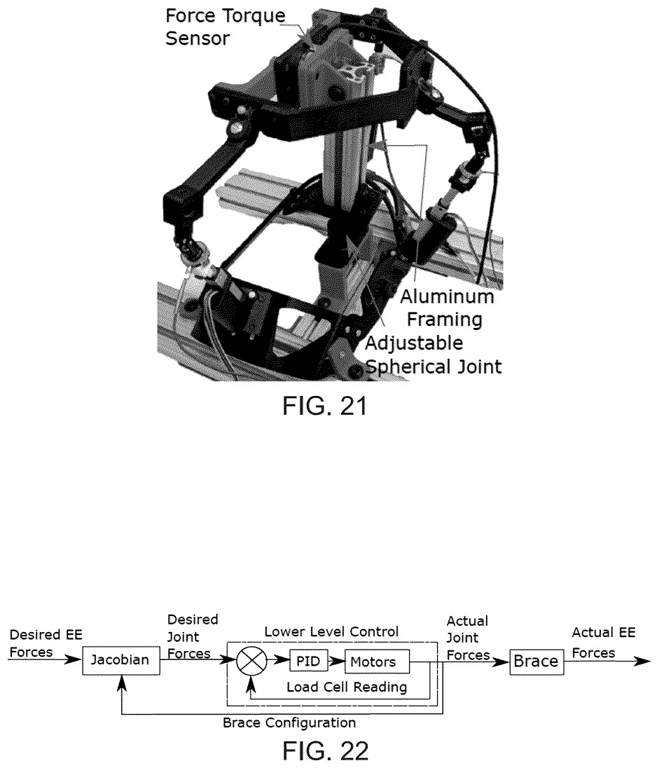

To validate the ability of the robotic brace 300 to provide a traction force to the head, the brace was placed in three different configurations, neutral, where flexion and lateral bending angles are set to 0, 10 degrees of flexion, and 5 degrees of lateral bending. These values of flexion and extension were chosen away from the neutral configuration while avoiding the extreme edges of the workspace. The force apparatus displayed in FIG. 21 was designed such that the end-effector 310 of the brace 300 could be configured to a certain position and orientation before being locked into place by the adjustable spherical joint. A piece of aluminum framing was used to fix the end-effector 310 in place. This testbed was designed to imitate human neck movement and validate the device's ability to apply forces in the direction and configurations commonly used during traction. An ATI Mini45 six-axis force torque sensor was connected between the end-effector 310 and the aluminum framing, allowing it to measure the forces exerted by the end-effector 310 on the environment. The origin and axes of the force torque sensor were aligned with those of the end-effector 310.

The control diagram in FIG. 22 depicts the method by which the upward force input was applied to the end-effector 310, with the lower level control once again occurring on the microcontroller. At each of the previously described positions, a vertical force of 22.3 N (5 lbs) was commanded for 15 s and then the commanded force was set to 0 N for 15 s, which was repeated three times. The remaining end-effector forces and moments were set to be zero. This method was chosen to mimic intermittent traction, which generally has a holding time of 10-25 s. This force pattern uniquely suits the brace as it requires the timed application and release of traction force.

During vertical force testing, the rise time, steady-state error, and maximum out-of-plane forces were collected. Rise time was calculated as the time required for the vertical force to reach its average steady state value from the time the command was sent. The steady-state error was computed as the difference between the commanded force and the average value of the vertical force during the second half of high force application. This timing was selected to allow the mechanism to reach a steady force. The maximum out-of-plane forces were the maximum forces measured in the x- and y-directions which were commanded to be zero.

### 4. Results and Discussion

The conducted experiments aimed to prove the ability of the neck brace 300 to achieve controlled position and orientation control, as well as vertical force control.

### A. Position Control

The maximum achieved angles for flexion and extension were 16.01° and −13.9°, respectively. This range of motion includes commonly used flexion angles, which can be around 15° during traction. The maximum lateral bending angles were 6.54° and −6.58° for left and right lateral bending, respectively. The flexion and extension angles achieved by the brace 300 is larger than the range of motion of other devices. The maximum lateral bending angle is comparable in the two designs with the maximum lateral bending angles of 7° in each direction. The brace 300 also achieved a maximum flexion range of 13.5°.

The brace's 300 ability to achieve flexed, laterally bent, and neutral positions demonstrates the device's ability to access different head orientations to apply traction forces. Despite the lateral bending angle being limited relative to other robotic devices, the independently controlled lateral bending and vertical translation of this device are key features that do not exist in existing mechanical traction devices.

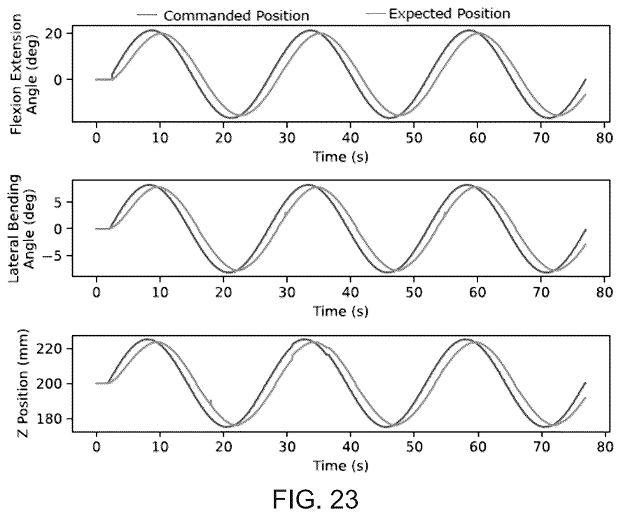

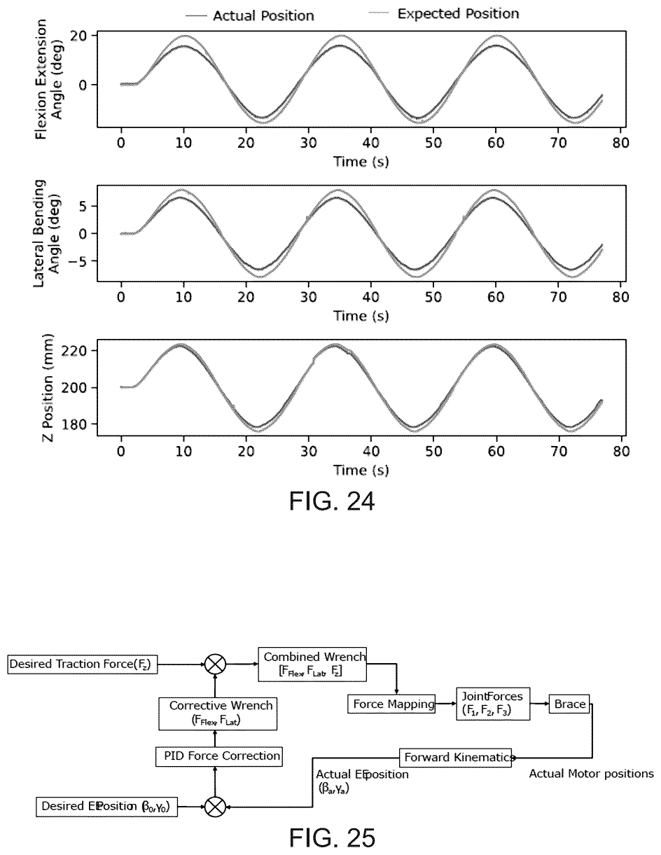

The ability of the motors to reach commanded positions was measured through the root-mean-square error (RMSE) between the commanded and expected end-effector position and orientation based on the motor encoders and the forward kinematics model. The RMSE, as displayed in Table I, remained below 5 degrees in both flexion-extension and lateral bending motion. FIG. 23 displays the two signals through the duration of each position trial. A major source of this error was the delay between when the command was sent to the microcontroller and when the motors achieved this position. This delay, likely caused by high gearing ratios, is not expected to negatively impact traction application, as the application happens gradually. The validity of the positional kinematic model can be evaluated by comparing the expected position and orientation of the end-effector 310 to the achieved position and orientation, which is shown in FIG. 24. The forward kinematics method described in Section 2 was used to calculate the expected end-effector orientation based on the recorded motor positions from the integrated encoders. The RMSE was selected as the main error measure between the commanded and measured positions.

TABLE I
ERROR BETWEEN COMMANDED (C), MEASURED
(M), AND EXPECTED (E) POSITIONS

| Trial | RMSE C vs E | RMSE E vs M |
|---|---|---|
| Flexion Extension | 4.72° | 2.41° |
| Lateral Bending | 1.97° | 1.04° |
| Vertical Translation | 6.03 mm | 1.56 mm |

### B. Force Control

The vertical force tests were comprised of a commanded upward traction force in a square wave. Therefore, the average rise times and steady-state errors, along with their respective standard deviations were calculated and are presented in Table II. The commanded force that was validated for vertical traction was 22.3 N (5 lbs), and it was able to achieve a mean force value of 17.17 N and 15.98 N in both the neutral and lateral bending configurations. In the flexed configuration, the brace 300 was able to reach 20.39 N.

TABLE II
VERTICAL FORCE CONTROL ANALYSIS

| | Rise | Steady State | Max Fₓ | Max F_{y} |
|---|---|---|---|---|
| Configuration | Time (s) | Error (N) | (N) | (N) |
| Neutral | 3.11 ± 1.64 | 5.07 ± 0.55 | −2.87 | 3.82 |
| Flexion | 4.48 ± 0.42 | 1.85 ± 0.29 | −3.60 | −1.92 |
| Lateral | 2.78 ± 0.08 | 6.26 ± 0.07 | 1.73 | 4.18 |

The variability of force application during manual traction drives the need for a robotic traction brace design. The low standard deviations in all three configurations tested (0.55 N, 0.29 N, 0.07 N) illustrate that the device can consistently apply a traction force when applied intermittently. The amount of cervical traction can be gradually applied to the maximum level tolerable by the patient. In this case, the exact force level is not as important as the ability to consistently deliver that same force during traction, especially during intermittent traction. As summarized in Table III, where the bottom row summarizes the device in this section, this mechanism combines several abilities in a way not seen in existing traction methods. The application of vertical force in this experiment demonstrates the first robotic neck brace 300 that can apply vertical force to the end-effector 310 in prescribed degrees of flexion and lateral bending.

TABLE III
CAPABILITIES OF TRACTION METHODS
AND ROBOTIC NECK BRACES

| | Independent | Independent | Controlled |
|---|---|---|---|
| | Vertical | Lateral | Force |
| System | Translation | Bending | Magnitude |
| Manual Traction | Y | Y | N |
| Mechanical Traction | Y | N | Y |
| 3RRS Device [18] | N | Y | Y |
| 3RPS Device [23] [22] | Y | N | Y |
| 3RPUR Device | Y | Y | Y |

The range of motion in flexion of the brace 300 is smaller than the full range of flexion angles, which may be resolved by changing the alignment of the base frame 320 and the shoulders. In the design described in this section, we have tested traction forces in the range of 20 N. In some embodiments, linear actuators, which can provide higher maximum forces will also be considered in the design. We can also increase the traction force magnitude by constructing the linkages out of a less flexible material such as aluminum. This will reduce the deformation of the linkages and allow higher forces to be applied by the end-effector 310. While the forces in the x- and y-directions during traction are low in comparison to the traction force itself, reducing link deformation may further minimize them.

The brace's 300 architecture has additional desirable features that can be explored. For users with anthropometric dimensions different than an average adult, the placement of the brace 300 on the human shoulders and parameters of the brace can be modified to allow for a shorter neck, narrower shoulders, and smaller head circumference. Additional degrees-of-freedom of the end-effector 310 can also be included based on the placement of the virtual points A. In addition, using the same chain structure, a 4 chain mechanism can allow two degrees-of-freedom to the end-effector 310. The architecture described in this section can be used and modified not only for traction applications, but also for other applications requiring specific control of head movement.

### 5. Conclusion

This section presented the design of an active robotic neck brace 300 that allows the end-effector 310 to have three degrees-of-freedom consisting of flexion/extension, lateral bending, and vertical translation. The novel 3RPUR mechanism provides independent vertical translation to the end-effector 310 that can be infeasible to achieve in other design architectures. This fills the previously unmet need for a traction device that can apply consistent traction forces and independently command the lateral bending angle. The validation of the position and force control of the end-effector 310 of this brace 300 allows potential future studies to apply traction forces on the human head. This brace 300 would enable clinicians to prescribe precise head orientations during activities of daily life such as sitting or standing. Additional capabilities, such as lateral bending and intermittent traction, provide an avenue for clinicians to explore new treatment methods. Benefits afforded by this brace 300 could improve clinical treatment and at-home pain relief for many people afflicted with cervical spine diseases.

### Section IV—Validation of the Section III Neck Brace

### 1. Introduction

The neck brace 300 described above in section III allows independent vertical force application as well as position control in the flexion-extension and lateral bending directions. Unlike traditional mechanical traction, it can achieve continuous flexion-extension and lateral bending angles, which have been validated in a benchtop study. The placement of these joints prevents axial rotation of the end-effector, allowing for the vertical motion to occur without being coupled to rotation of the head.

The neck brace 300 provides traction capabilities previously unavailable such as the ability to independently control the vertical force in multiple positions of flexion and lateral bending. To validate the ability of the brace to apply controlled cervical traction in different positions to human users, nine healthy young adults were recruited to use the brace. Using the integrated motor encoders and external motion capture, the positioning accuracy and slip between the end-effector and the user's head was measured. The force application abilities of the brace were validated using the load cells in each chain. In five different head orientations (neutral, flexed 10°, flexed 15°, and laterally bent to the left and right 5°), three different force magnitudes (4 N, 11 N, 22 N) were applied to the head. The overall force error was low for the low and medium force conditions, with the error remaining below 1 N and 3 N, respectively. The force applied also did not deviate more than 0.7 N from the steady-state value, demonstrating consistent traction force. Both the slip between the end-effector and the head and the positioning accuracy of the control strategy were found to be less than 2° and 1°, respectively. Finally, using electromyography, we show the ability of the brace to affect muscle activation, consistent with other forms of traction. Between three levels of traction forces, the splenius capitis muscles showed significant differences in muscle activation, increasing activation with increased force. The positioning and force application validation of the robotic traction neck brace confirms the ability of the device to apply controlled vertical forces to the head in controlled positions of flexion and lateral bending. This paves the way for future studies using this device to investigate new ways to assist people with cervical spine deformities.

In a previous benchtop study using external motion capture and an external force-torque sensor, the consistency of force application as well as the accuracy of the position control were validated. In this section IV, the ability of the neck brace 300 to apply vertical forces and maintain a specific position is validated with human subjects. This section aims to quantify how well the brace constrains the head while traction forces are being applied, verify the consistency of force application, and provide insights into the way muscle activation changes during traction.

### 2. System Design

The hardware design changes made to accommodate human users did not affect how the kinematics was calculated, but moving the side joints at the end-effector such that they were closer to the rear joint increased the range of motion of the brace in the flexion direction. Although the strap placements were modeled after the halter device, rigid links were used along with these fabric straps to preserve the brace's ability to control the head position. These links were adjustable, and the straps could be tightened to provide a secure fit for each user despite differing head sizes.

A common characteristic of limited-degree-of-freedom mechanisms is the coupling of components of the end effector wrench. This is also true of the 3-RPUR mechanism used in this section. The mapping between the angular velocities and the linear velocities yields the velocity constraint matrix G, where gᵢᵢ is a configuration-dependent variable.

$$[ \begin{matrix}{ω}_{1} \\ {ω}_{2} \\ {ω}_{2}\end{matrix} ] = {[ \begin{matrix}0 & {g}_{12} & 0 \\ {g}_{21} & {g}_{22} & 0 \\ {g}_{31} & {g}_{32} & 0\end{matrix} ]}_{\underbrace{G}} [ \begin{matrix}\dot{x} \\ \dot{y} \\ \dot{z}\end{matrix} ] \qquad ( 1 )$$

$$[ \begin{matrix}\begin{matrix}{F}_{1} & {F}_{2}\end{matrix} & {F}_{3}\end{matrix} ] = \qquad ( 2 )$$

The column of zeros in the rightmost column of matrix G in (1) allows for independent force control in the vertical direction, because the vertical force (F_{z}) will not be coupled with any other component of the wrench before being mapped to the joint forces in (2). Therefore, the 3-RPUR architecture creates a mapping between the joint and end-effector forces which keeps the vertical force at the end-effector separate from the other components of the wrench.

### A. Control Strategy

In this section IV, we build upon the ability of the mechanism to independently control the vertical force by utilizing the other two components of the coupled wrench. The other components of the wrench apply forces with the goal of correcting any positional deviation in the flexion-extension and lateral bending directions.

The following equation completes the mapping in 2 to allow us to inspect each component of the coupled wrench.

$$[ \begin{matrix}\begin{matrix}{F}_{1} & {F}_{2}\end{matrix} & {F}_{3}\end{matrix} ] = {[ \begin{matrix}{F}_{x} + {g}_{21} {M}_{y} + {g}_{31} {M}_{z} \\ {F}_{y} + {g}_{12} M \text{?} + {g}_{22} {M}_{y} + {g}_{32} M \text{?} \\ {F}_{z}\end{matrix} ]}^{T} J \qquad ( 3 )$$

$$\text{?} \text{indicates text missing or illegible when filed}$$

In 3, there are three elements of the coupled wrench, which are then multiplied by the Jacobian to get the individual forces

Within the workspace, the values of all elements of the matrix G were inspected. It was observed that the values of g₃₁, g₂₂, g₃₂ are consistently at least one order of magnitude lower than the remaining non-zero values of the G matrix. Therefore, upon inspection of (3), an estimation can be made that the first term is primarily related to lateral bending, as the terms that affect it the most are the force and moment in the lateral bending direction. Similarly, the second term is mainly governed by the force and moment in the flexion-extension direction.

This allows the controller in FIG. 25 to send corrective forces in the first and second terms of the wrench command to affect lateral bending and flexion-extension motion, respectively. The corrective wrench is a three-element array that is computed based on the deviation from the commanded orientation and the controller gains (K_{γ},K_{β}). It is then summed with the original F_{Z} command, resulting in the following mapping.

$$[ \begin{matrix}\begin{matrix}{F}_{1} & {F}_{2}\end{matrix} & {F}_{3}\end{matrix} ] = [ \begin{matrix}{K}_{γ} ( {γ}_{err} ) & {K}_{D} ( {β}_{err} ) & F \text{?}\end{matrix} ] J \qquad ( 4 )$$

$$\text{?} \text{indicates text missing or illegible when filed}$$

where βₑᵣᵣ is the flexion-extension angular error and γₑᵣᵣ is the lateral bending angular error.

In this way, the force corrections necessary for maintaining a specific position can be included in the control law and may be incorporated simultaneously with the existing force controller.

### 3. Experimental Procedures

Nine subjects with an average age of 25.6±3.3 years who had no history of neck pain or neck injury participated.

### A. Experimental Protocol

Electromyography (EMG) sensors (Noraxon Inc.) were affixed to three different muscle groups: the sternocleidomastoid (SCM), the splenius capitis (SC), and the upper trapezius (UT). Motion capture markers were also placed at three different bodies during the experiment. One set was placed at the brace end-effector. Another four markers were placed on the face at the forehead, left and right cheeks and chin. Finally, markers were placed at the base frame on the shoulder section of the brace. The brace sensor data, VICON motion capture information, and EMG recordings were synced during recording and then processed offline.

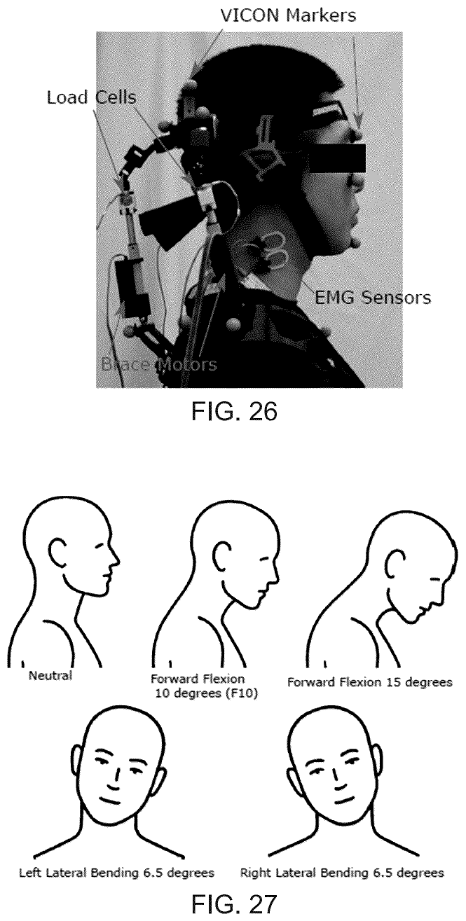

The experiment protocol consisted of several stages: i) fitting, ii) tuning, and iii) force application. During the fitting stage, the brace was secured to the participant using shoulder straps such that there was minimal movement between the shoulders and the base of the brace. The end effector was then fitted to the participant's head as depicted in FIG. 26, which shows a user wearing the neck brace 300 during the traction experiment, with Vicon markers positioned at the face, end-effector, and shoulders. The straps were tightened such that slippage was minimized, but not so tightly that it caused pain to the wearer. Then, during the tuning stage, the proportional, integral, and derivative (PID) control used for positional correction was tuned. The integral and derivative gains were set to zero, and the proportional gain was gradually increased until stable oscillations of the position could be observed. Using the Ziegler-Nichols tuning method, the proportional, integral, and derivative terms were set for the control law. These terms were used throughout the experiment for each subject.

Each participant was placed in each of the five positions by the brace: Neutral position (N): (0° Flexion, 0° Lateral Bending), 10° flexion (F10), 15° of flexion (F15), 5° Left Lateral Bending (LatL), 5° Right Lateral Bending (LatR), displayed in FIG. 27. Traction was applied while the wearer was seated. The motors on the brace moved the end-effector attached to the head to the desired orientation. Once the wearer was placed into this position and orientation, they were asked to follow the movement of the brace. The brace was then commanded to apply an upward force to the head. The forces were 4.45 N (1 lb), 11.12 N (2.5 lb), and 22.24 N (5 lb), with three trials completed per force level. The force profile was a 5-second ramp from zero force to the desired constant force, after which the constant force would be applied for 10 seconds.

### B. Data Analysis

1) Brace Data: Validation of the new hybrid control strategy required the analysis of the position and orientation of the end-effector as well as the force being applied by the end-effector on the head. Using the motor encoders in the brace as well as the load cells, the expected end-effector position and the expected force at the end-effector were computed.
2) EMG Data Processing and Statistical Analysis: The analysis of the EMG data began with the following steps: (i) remove the DC offset, (ii) band pass filter signal between 10-400 Hz, (iii) rectify signal, (iv) employ root mean square with a 20 ms envelope, and (v) normalize to the highest value in each channel throughout the entire experiment. Each traction application trial was segmented into ramp time (when the force gradually increased from zero to the desired force values) and constant force. The integrated EMG (iEMG) values were extracted from the constant force range.
3) Statistical Analysis: The statistical tests used to analyze the EMG data involved a force-wise comparison and position-wise comparisons. The three forces were compared against each other, pooling all positions. Omnibus tests were carried out, and when significance was found (p 0.05), post-hoc pairwise tests were conducted using a Holm-Sidak correction.

### 4. Results and Discussion

The experimental goals of this study are related both to the validation of the novel neck brace in human subjects and to the exploration of muscular responses to traction. The first goal is to validate the brace's ability to simultaneously control both the vertical force and end-effector orientation. The second goal is to explore how the muscles around the neck respond to differences in i. force magnitude and ii. head orientation.

### A. Force Validation

Two main variables regarding the brace's ability to apply the desired force were investigated: the steady-state error of the force application and the steady-state deviation, which is a measure of how much the applied force deviates from its steady-state value. The steady-state error characterizes the brace's ability to achieve the commanded force, while the steady-state deviation characterizes the ability of the brace to keep the force constant throughout the force application.

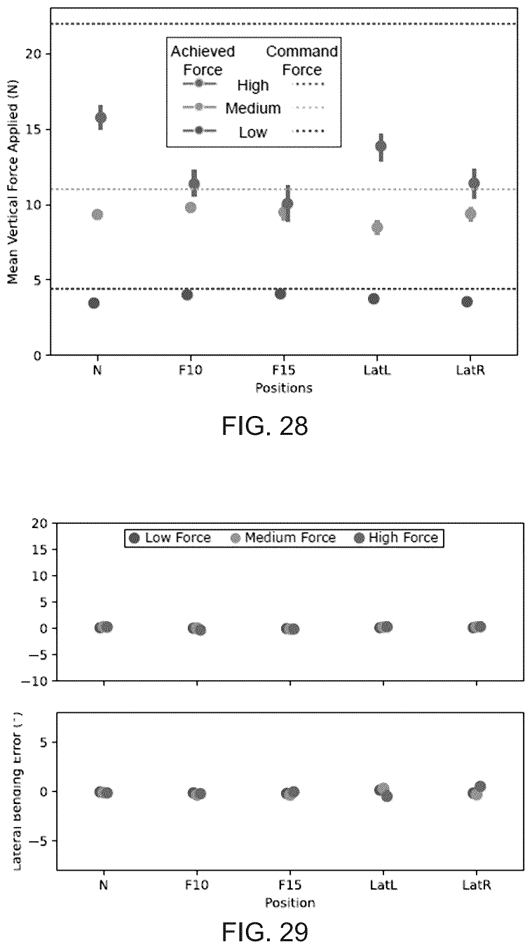

In FIG. 28, the achieved forces and commanded forces are displayed separated by both the magnitude of the commanded force and the position of the end-effector. These data points are the compiled data from all subjects and trials. The standard deviation is also plotted to display how much the applied force varies across subjects and trials. In the lowforce condition, the error remained below 1 N in all positions, and in the medium-force condition, the error remained below 2 N in most of the positions.

The steady-state deviation, displayed in Table I, remained below 0.7 N even in the highest force condition. Furthermore, in the low and medium force conditions, the deviation is even lower, remaining below 0.5 N. The very low force deviation observed in the brace validates the ability of the device to keep a steady force with a human user. Among all devices able to apply traction to the head, this demonstrates the first to be able to repeatably apply consistent forces to the headneck in both flexion and lateral bending.

TABLE I
Vertical Force SSD (N)

| Force | Neutral | F10 | F15 | LatL | LatR |
|---|---|---|---|---|---|
| Low | 0.34 | 0.39 | 0.44 | 0.49 | 0.29 |
| Med | 0.31 | 0.36 | 0.32 | 0.27 | 0.28 |
| High | 0.45 | 0.55 | 0.67 | 0.50 | 0.34 |

### B. Position Validation

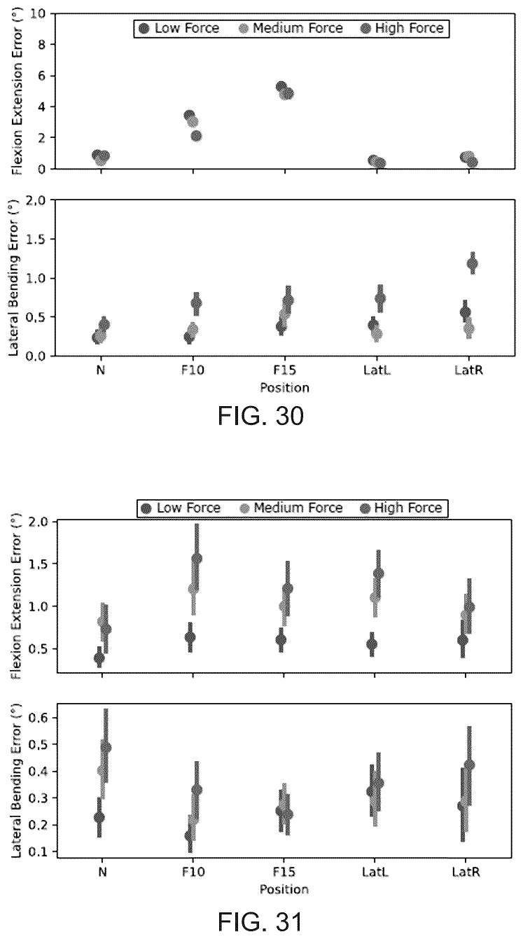

To analyze the ability of the brace to adequately control the position of the head, several sources of data were used. First, the integrated motor encoder information was used, along with forward kinematics, to verify the ability of the proposed force corrective control to keep the end-effector in the correct position. Then, the VICON markers at the end-effector attached to the brace were used to compute the achieved position of the end-effector. Finally, two groups of markers, the markers on the face, and the markers on the end-effector were compared against each other. Ideally, the position and orientation of the two bodies relative to each other would remain fixed, but movement between the robot and the user is common in wearable devices and should therefore be quantified. By separating the position validation into these three factors, it allows us to understand how much error each part of the system is contributing: the controller error, any mechanical or fabrication errors in the brace, and the slip between the end-effector and the head.
1) Position Correction Validation: When analyzing the position of the end-effector as measured by the motor encoders, the error remains very low in all positions and forces. These errors are presented in FIG. 29 where all subjects and trials were included. The mean error recorded throughout the duration of the force application remained below 1 degree in both the flexion-extension direction and the lateral bending direction. This very low positional error confirms that the force correction to positional error described in Section 2 is a valid way to control the position of the end-effector during traction. The simplification of the matrix G allows us to take advantage of the unique structure of the mechanism to input the corrective forces to the wrench command. Furthermore, due to the low error across positions, we may conclude that the force corrective control strategy is capable of controlling the position of the end-effector in a variety of positions, both close and far from the neutral position.
2) End-Effector Position Validation: Using the markers on the brace end-effector, the orientation of the brace can be externally validated. The markers at the brace end-effector formed one rigid body, and the markers at the shoulders formed another. The angles between the two bodies were computed, and the error between these values and the commanded position values is presented in FIG. 30. In the flexion-extension direction, the error between the commanded value and the achieved value increased as the commanded flexion angle increased. In the position furthest from the Neutral position, the error is, on average, 5°. The end-effector positioning error increased as the commanded position was further from the neutral position. The overall error was only slightly higher than in the single-plane position control benchtop testing, but the reduced accuracy of positioning of the head may be due to the deformation of the links and straps during force application. Furthermore, the trend of error increasing as the position ventures further from the neutral has been observed in existing neck braces.
3) Slip Characterization: The slip between the brace end-effector and the face was computed using the facial markers and the markers attached to the end-effector. The relative position and orientation of these bodies relative to each other were computed before traction was applied. Then, during traction application, any change to that relative orientation was recorded as well as the direction in which the change occurred. The amount of slip in the flexion-extension and lateral bending directions were compiled among all subjects and trials and are presented in FIG. 31, where they are separated by force level and position. Overall, slip between the end-effector and the head remained below 2° in the flexion-extension direction and 0.7° in the lateral bending direction. The brace performs comparably with existing neck braces, where slip was on average 0.15-1.39°.

Note that in this study, the traction device is actively applying forces to the user's head vertically, a direction the user cannot naturally follow. Despite this, the orientation of the head exhibits minimal slip relative to the end-effector. The larger forces applied by this device to the head in the vertical direction does not affect the positioning of the head, which is a critical aspect of the brace's performance. The low slip between the head and the end-effector also validates the method by which the end-effector is attached to the head, using straps under the chin and base of the skull.

The slip recorded during the low and medium forces is lower than that of existing robotic neck braces, which may be due to a combination of the lack of dynamic motion asked of the user and the method by which the end-effector is attached to the head. Instead of relying on a single headband around the forehead, additional straps around the chin and occiput were used keep the head securely attached to the end-effector, as best seen in FIG. 26. This control of the head means the device is able to hold the head steady during traction, which is critical to traction devices, as uncontrolled movement of the head during traction can cause injury to the user.

### C. Muscle Activation

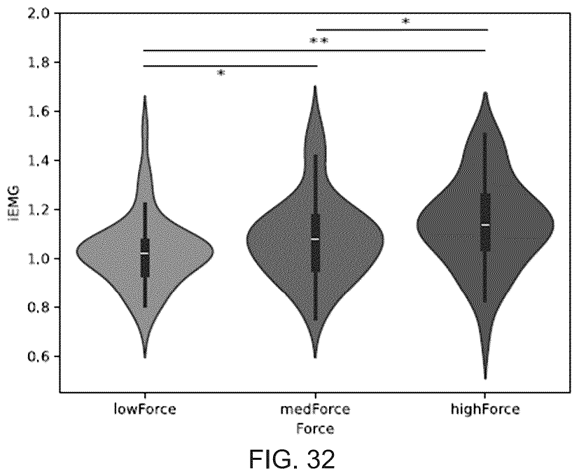

During traction, the muscle activations in the splenius capitis muscle increased as the amount of force increased, even when observing all positions. The EMG values during low force were 2.3% higher than the no-force condition, the medium force was 7.8% higher, and the high force was 13.9% higher. All of these forces were significantly different from each other, with p<0.05, p<0.05, and p<0.01 for the differences between low and medium, medium and high, and low and high, respectively. FIG. 32 depicts the Splenius Capitis muscle activation during steady-state force application. The forces are normalized against no force condition *:p<0.05, **:p<0.01.

TABLE II
Muscle group activity normalized to a no force baseline.

| Position | UT Sum | SC Sum | SCM Sum |
|---|---|---|---|
| Neutral | 0.84 (0.12) | 1.14 (0.13) | 1.02 (0.32) |
| F10 | 0.91 (0.33) | 1.07 (0.16) | 1.20 (0.42) |
| F15 | 0.98 (0.56) | 1.18 (0.27) | 1.01 (0.33) |

UT = upper trapezius,
SC = splenius capitis,
SCM = sternocleidomastoid

The muscle activation patterns in SC agree with the findings in the literature. As the amount of force increases, the activation of the splenius capitis muscles also increases. The absence of significant upper trapezius changes across the forces also agrees with the results in the literature. These findings serve to validate the ability of the brace to apply traction to the cervical spine in a physiologically measurable way. The findings related to the muscular response to traction serve to underscore how muscular activation can be affected by cervical traction. Even with forces lower than the traditional traction forces, we see significant changes in the splenius capitis muscle activations.

Overall, the brace was able to deliver accurate forces to the head in both the medium and low force conditions. However, in one of the three modes (high force), there was a higher error. The error in the high force condition was largely caused by limitations in the workspace of the mechanism. In other embodiments, enlarging the vertical workspace of the robot by using motors with larger stroke lengths should allow for higher forces to be applied. A larger workspace should also reduce end-effector angular error by keeping the head further from the edges of the workspace.

The validation of the brace's ability to simultaneously control the position and vertical force applied to the head creates more opportunities for utilizing the unique capabilities of the robotic traction brace. Different force and position profiles during traction, and incorporation of EMG biofeedback can also be used to augment cervical traction when treating cervical disk degeneration.

### 5. Conclusion

This section IV demonstrates the ability of the brace 300 to apply controlled vertical forces to the head while keeping the head in specified positions of flexion and lateral bending. Through this experiment, we were able to validate the performance of the robotic traction brace with healthy participants. Motion capture analysis confirmed that the head stays in a controlled position during traction in a neutral position. Furthermore, the neck brace was able to replicate findings regarding muscle activation patterns in the splenius capitis muscles from a different cervical traction device. The ability to simultaneously control both position and vertical force offers a variety of new opportunities to utilize different head orientations to affect cervical traction.

### Section V—Conclusion

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A head-neck traction brace comprising:
a base frame configured to be supported by a subject's shoulders;
an end-effector frame configured so that it can be removably attached to the subject's head; and
four chains of joints, each of which has a lower revolute joint connected to the base frame followed by a prismatic joint followed by a universal joint followed by an upper revolute joint,
wherein the upper revolute joint is connected to the end-effector frame.

2. The traction brace of claim 1, further comprising a plurality of linear actuators, each of which is configured to actively control a respective one of the prismatic joints.

3. The traction brace of claim 1, further comprising two linear actuators, each of which is configured to actively control a respective lateralmost one of the prismatic joints.

4. The traction brace of claim 3, wherein all of the prismatic joints that are not actively controlled by one of the linear actuators are passive prismatic joints.

5. The traction brace of claim 1, wherein the universal joint comprises two revolute joints.

6. The traction brace of claim 1, further comprising at least one additional chain of joints having a first end connected to the base frame and a second end connected to the end-effector frame.

7. A head-neck traction brace comprising:
a base frame configured to be supported by a subject's shoulders;
an end-effector frame configured so that it can be removably attached to the subject's head; and
three chains of joints, each of which has a lower revolute joint connected to the base frame followed by a prismatic joint followed by universal joint followed by an upper revolute joint connected to the end-effector frame.

8. The traction brace of claim 7, further comprising a plurality of linear actuators, each of which is configured to actively control a respective one of the prismatic joints.

9. The traction brace of claim 7, further comprising two linear actuators, each of which is configured to actively control a respective lateralmost one of the prismatic joints.

10. The traction brace of claim 9, wherein all of the prismatic joints that are not actively controlled by one of the linear actuators are passive prismatic joints.

11. The traction brace of claim 7, wherein a plurality of revolute joints are used to implement each of the universal joints.

12. The traction brace of claim 7, further comprising a fourth chain of joints having a lower revolute joint connected to the base frame followed by a prismatic joint followed by universal joint followed by an upper revolute joint connected to the end-effector frame.

13. A head-neck traction brace comprising:
a base frame configured to be supported by a subject's shoulders;
an end-effector frame configured so that it can be removably attached to the subject's head; and
four chains of joints, each of which has a lower revolute joint that is mounted to the base frame followed by a middle revolute joint followed by a universal joint followed by an upper revolute joint that is mounted to the end-effector frame.

14. The traction brace of claim 13, further comprising a plurality of rotary motors, each of which is configured to actively control a respective one of the lower revolute joints.

15. The traction brace of claim 13, further comprising two rotary motors, each of which is configured to actively control a respective lateralmost one of the lower revolute joints.

16. The traction brace of claim 15, wherein all of the lower revolute joints that are not actively controlled by one of the rotary motors are passive revolute joints.

17. The traction brace of claim 13, further comprising at least one additional chain of joints having a first end connected to the base frame and a second end connected to the end-effector frame.

18. A head-neck traction brace comprising:
a base frame configured to be supported by a subject's shoulders;
an end-effector frame configured so that it can be removably attached to the subject's head; and
four chains of joints, each of which has a respective first end connected to the base frame and a respective second end connected to the end-effector frame,
wherein each of the four chains of joints has either (a) a lower revolute joint followed by a prismatic joint followed by a universal joint followed by an upper revolute joint or (b) a lower revolute joint followed by a middle revolute joint followed by a universal joint followed by an upper revolute joint.

19. The traction brace of claim 18, wherein each of the lateralmost chains of joints has a lower revolute joint connected to the base frame followed by a prismatic joint followed by a universal joint followed by an upper revolute joint connected to the end-effector frame, and
wherein the traction brace further comprises two linear actuators, each of which is configured to actively control a respective lateralmost one of the prismatic joints.

20. The traction brace of claim 18, wherein each of the lateralmost chains of joints has a lower revolute joint that is mounted to the base frame followed by a middle revolute joint followed by a universal joint followed by an upper revolute joint that is mounted to the end-effector frame, and
wherein the traction brace further comprises two rotary motors, each of which is configured to actively control a respective lateralmost one of the lower revolute joints.

21. The traction brace of claim 18, further comprising at least one additional chain of joints having a first end connected to the base frame and a second end connected to the end-effector frame.
